(19) United States
(12) Patent Application Publication
(10) Pub. No.: US 2024/0376437 A1
(43) Pub. Date: Nov. 14, 2024

(54) TARGETING VON WILLEBRAND FACTOR TO MODEL DISEASE IN HUMAN PLURIPOTENT STEM CELLS

(71) Applicant: University of Washington, Seattle, WA (US)

(72) Inventors: Hongxia Fu, Seattle, WA (US); Benjamin Freedman, Seattle, WA (US); Jose A. Lopez, Seattle, WA (US)

(73) Assignee: University of Washington, Seattle, WA (US)

(21) Appl. No.: 18/444,289

(22) Filed: Feb. 16, 2024

Related U.S. Application Data
(60) Provisional application No. 63/464,695, filed on May 8, 2023.

(51) Int. Cl. C12N 5/071; C12M 3/00; C12M 3/06; C12N 9/22; G01N 33/50

(52) U.S. Cl. CPC C12N 5/069; C12M 21/08; C12M 23/16; C12N 9/22; G01N 33/5064; C12N 2310/20; C12N 2506/02; C12N 2506/45; C12N 2513/00

(57) ABSTRACT

The present disclosure provides methods and compositions useful for generating cellular models of VWF disease from human pluripotent stem cells. In one aspect, the present disclosure relates to a method for the generation of an in vitro cellular model of VWF disease comprising VWF disease-relevant endothelial cells derived from human pluripotent stem cells (hPSCs). In an embodiment, the human pluripotent stem cells (hPSCs) are treated with an agent effective in suppressing or deleting at least one gene sequence encoding a protein and/or a subunit thereof, where the protein and/or subunit thereof is associated with VWF-linked secretion. The present disclosure also provides uses of the cellular models disclosed herein.

### CROSS-REFERENCE TO RELATED APPLICATION

The present application claims the benefit of Provisional Application No. 63/464,695, filed May 8, 2023, the entire disclosure of which is hereby incorporated by reference herein for all purposes.

### STATEMENT OF GOVERNMENT LICENSE RIGHTS

This invention was made with government support under Grant Nos. W81XWH-21-1-0006 and W81XWH-21-1-0007, awarded by the Department of Defense and Grant No. R35HL145262, awarded by the National Heart, Lung, and Blood Institute and Grant Nos. K25HL135432 and R01DK117914 and U01DK127553, awarded by the National Institutes of Health. The government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 3915-P1335USUW_Seq_List_20240216.xml. The XML file is 63,989 bytes; was created on Feb. 16, 2024; and is being submitted electronically via Patent Center with the filing of the specification.

### BACKGROUND

A functional vasculature, lined with endothelial cells (EC) and carrying flowing blood, is an essential component for diverse vertebrate tissues and organs. The vasculature has evolved an elaborate system of hemostasis to rapidly stop bleeding at sites of injury. This system must be delicately balanced and controlled to sustain life.

Von Willebrand factor (VWF), an essential blood protein in hemostasis and thrombosis, is synthesized in EC or megakaryocytes and secreted as multimers into the circulatory system. VWF is stored in rod-shaped, EC-specific organelles, known as Weibel-Palade bodies (WPBs). Upon stimulation, VWF is secreted from WPBs to enter the blood plasma or to form elongated ‘strings’ attached to the EC surface. There, VWF multimers can extend under flow and become activated to bind with platelets to initiate thrombus formation. Deficient or defective VWF causes the world's most common bleeding disorder, Von Willebrand disease (VWD), which affects one in ˜100 people worldwide. VWD can be treated by either administration of desmopressin to increase VWF release from EC or transfusion of VWF in blood plasma. In contrast to VWD, VWF gain of function resulting from genetic mutations or pharmacological interventions can cause life-threatening thrombosis, which is a concern in many diseases such as cardiovascular disorders and transplantation. Understanding and analyzing von Willebrand factor in relation to endothelial cells, protein pathways linked to VWF secretion, and disease modeling is important to create drugs and therapies that will ultimately help treat and cure these bleeding disorders.

Although there has been progress in developing VWF-deficient models such as knockout animals or blood outgrowth EC (BOEC) to study the role of VWF in disease, challenges remain in the use of these primary human EC for disease modeling and regenerative medicine applications, including donor-to-donor variability, short passage number, difficulty in sourcing of the cells, and limited differentiation potential.

### SUMMARY

To overcome the above limitations, aspects of the present disclosure include methods and compositions related to production of human Pluripotent Stem cells (hPSCs) for generation of cellular models of Von Willebrand Factor (VWF) disease, use of such models as tools for elucidating effects of VWF deficiency on endothelial cell (EC) differentiation and function, and identifying agents that regulate/modulate VWF expression, storage, and secretion in hPSC-ECs.

Accordingly, in one aspect, the present disclosure provides a method of generating a cellular model of Von Willebrand Factor (VWF) disease. In some embodiments, the method comprises (i) providing at least one pluripotent stem cell; (ii) contacting the at least one pluripotent stem cell with an agent effective in suppressing or deleting at least one gene sequence or a portion thereof encoding a protein or a subunit thereof, wherein the protein or a subunit thereof is associated with VWF-linked protein secretion; and (iii) differentiating said pluripotent stem cell into an endothelial cell. In an embodiment, the at least one gene sequence or a portion thereof comprises the VWF gene or a portion thereof. In some embodiments, the agent is effective in disrupting the VWF gene to produce a pluripotent stem cell-derived endothelial cell with a knock out mutation of VWF. In certain embodiments, the agent effective in disrupting the VWF gene comprises a CRISPR/Cas9 genome editing system comprising nucleotide sequences encoding CRISPR-Cas guide RNAs. In some embodiments, the guide RNAs hybridize with a target sequence in exon 1 or in exon 2 of the VWF gene (SEQ ID NO: 1). In an embodiment, the CRISPR-Cas guide RNAs are selected from SEQ ID NO: 2 or SEQ ID NO: 3.

The present disclosure also provides an endothelial cell deficient in VWF (von Willebrand factor gene). In some embodiments, the endothelial cell deficient in VWF (von Willebrand factor gene) is produced by the methods disclosed herein.

In another aspect, provided herein is an in vitro generated human endothelial cell differentiated from a human pluripotent stem cell (hPSC-EC). In some embodiments, the hPSC-EC expresses CD144, CD31, P-selectin, and ANG2. In some embodiments, the hPSC-EC expresses VWF stored in WPBs. In a related embodiment, the hPSC-EC forms capillary-like structures. In some embodiments, the hPSC-EC secretes VWF multimers.

In an embodiment, the hPSC-EC is deficient in at least one gene sequence associated with VWF-linked protein secretion. In some embodiment, the hPSC-EC lacks the VWF (von Willebrand factor gene). In an embodiment, the hPSC-EC lacking VWF has a morphology similar to a human pluripotent stem cell differentiated into endothelial cell (hPSC-EC) comprising VWF. In some embodiments, the hPSC-EC lacking VWF forms capillary-like structures with similar tube length and branch numbers as formed by hPSC-EC comprising VWF. In some embodiments, the hPSC-EC lacking VWF expresses CD144, CD31, P-selectin, and ANG2 in similar quantities and in similar localization patterns as expressed by hPSC-EC comprising VWF. In yet another embodiment, the hPSC-EC lacking VWF does not secrete VWF multimers.

In some embodiments of the present disclosure, the hPSC-ECs disclosed herein are capable of forming a cell line, a teratoma, a tissue, or an organoid.

In yet another aspect, the present disclosure provides a cell line generated from at least one hPSC-EC disclosed herein. In some embodiments, the cell line models a feature of blood disorder selected from thrombosis, deep vein thrombosis, blood clots, stroke, heart disease and von Willebrand disease. The present disclosure also provides an organoid generated from at least one hPSC-EC disclosed herein. In some embodiments, the organoid models a feature of blood disorder selected from thrombosis, deep vein thrombosis, blood clots, stroke, heart disease and von Willebrand disease.

In yet another aspect, the present disclosure relates to a microfluidic culture device comprising cells obtained from the one or more cell lines disclosed herein. In some embodiments, the present disclosure provides a microfluidic culture device comprising at least one organoid disclosed herein. In some embodiments, the microfluidic culture device is an organ-on-chip model configured to represent VWF disease. In an aspect, the present disclosure relates to a system for cultivation, maintenance, and/or analysis of one or more cell lines, teratomas, tissue constructs, or an organoid, or a group of cells generated from the hPSC-ECs disclosed herein.

In yet another aspect, the present disclosure relates to a high throughput method for identifying a therapeutic agent for treating a bleeding disorder. In some embodiments, the method comprises contacting a plurality of candidate agents with cells obtained from at least one of the cell lines disclosed herein. In some embodiments, the method comprises contacting a plurality of candidate agents with the at least one organoid comprised in the microfluidic device disclosed herein. In some embodiments, the microfluidic device comprises an organ-on-a-chip configured to represent VWF disease. In some embodiments, the agent effective in enhancing the expression levels of VWF; and/or restoring the expression of VWF; and/or restoring VWF linked protein secretion; and/or restoring VWF string formation, is identified as the therapeutic agent effective for treating bleeding disorders.

In some embodiments, the therapeutic agent is selected from a genetic agent, a component of the cell culture environment, or a small molecule agonist of VWF expression. In certain embodiments, the therapeutic agent is a genetic agent. In some embodiments, the genetic agent is selected from a vector comprising a polynucleotide encoding VWF protein, a subunit thereof, or a derivative thereof, and a polynucleotide encoding a protein linked with VWF secretion, a subunit thereof, or a derivative thereof. In some embodiments, the therapeutic agent is a component of the cell culture environment. In certain embodiments, the component of the cell culture environment is blood plasma. In some embodiments, the therapeutic agent is a small molecule agonist of VWF expression. In certain embodiments, the small molecule agonist of VWF expression comprises DAPT. In some embodiments, the method further comprises assessing toxicity of the therapeutic agent. In some embodiments, the method further comprises assessing clearance and pharmacokinetics of the therapeutic agent. In some embodiments, the bleeding disorder is selected from thrombosis, deep vein thrombosis, blood clots, stroke, heart disease and von Willebrand disease.

In another aspect, the present disclosure also provides a biochemical assay comprising the cells obtained from at least one cell line disclosed herein and/or the at least one organoid comprised in the microfluidic device disclosed herein. Also disclosed herein is a kit comprising a cell line, an organoid, or a microfluidic device disclosed herein.

In yet another aspect, the present disclosure pertains to a composition comprising at least one human pluripotent stem cell differentiated into an endothelial cell (hPSC-EC). In some embodiments, the hPSC-EC is deficient in at least one gene sequence associated with VWF-linked protein secretion. In some embodiments, the hPSC-EC lacks the VWF (von Willebrand factor gene).

In yet another aspect, provided herein is a method of generating one or more human pluripotent stem cell lines. In some embodiments, the method comprises (i) identifying at least one protein linked to VWF-linked protein secretion in a biological sample; (ii) contacting a plurality of hPSCs with an agent effective in suppressing or deleting at least one gene sequence or a portion thereof encoding the identified protein or a subunit thereof; (iii) disrupting a VWF-linked protein secretion pathway in the plurality of hPSCs to obtain a plurality of modified hPSCs; and (iv) deriving a cell lineage or tissue from the modified hPSCs to obtain human pluripotent stem cell lines, wherein endothelial cells are produced. In some embodiments, the biological sample comprises blood. In some embodiments, the one or more human pluripotent stem cell line is deficient in VWF-linked protein secretion.

In some embodiments, the method further comprises an additional step of stimulating endothelial cell specific organelles. In some embodiments, the specific organelles are Weibel-Palade bodies (WPBs). In some embodiments, the identified protein is VWF. In some embodiments, the VWF is identified using antibodies specific to a multi-lineage phenotype. In some embodiments, the disrupting step changes an amount of VWF-linked protein secretion. In some embodiments, the gene sequence encodes VWF protein (SEQ ID NO: 4). In some embodiments, the suppressing or deleting step is performed by gene editing. In some embodiments, the method produces VWF-deficient mutants. In an embodiment, the one or more human pluripotent stem cell line models a feature of blood disorder selected from thrombosis, deep vein thrombosis, blood clots, stroke, heart disease and von Willebrand disease. In some embodiments, the method further comprises the step of generating non-mutant isogenic human pluripotent stem cell derived endothelial cell (hPSC-EC) control cell lines with intact VWF linked protein secretion. In some embodiments, the cell line is used in a biochemical assay or a pharmaceutical kit. In some embodiments, the cell line comprises somatic cells.

In an aspect, the present disclosure relates to an organoid comprising one or more human pluripotent stem cell derived endothelial cells deficient in VWF-linked protein secretion. In another aspect, the present disclosure relates to an organoid comprising one or more human pluripotent stem cell derived endothelial cells comprising intact VWF-linked protein secretion.

In another aspect, the present disclosure relates to a microfluidic culture device comprising cells obtained from the cell line or the organoid disclosed herein. In some embodiments, the device comprises an organ-on-a-chip culture device configured to represent VWF disease.

In yet another aspect, the present disclosure provides a method of treating a bleeding disorder in a subject in need thereof, the method comprising administering to the subject an effective amount of a therapeutic agent identified by the methods of the present disclosure. In an embodiment, the agent is effective in restoring VWF string formation.

### DESCRIPTION OF THE DRAWINGS

This patent or application file contains at least one drawing executed in color. Copies of this patent or patent application publication with color drawing(s) will be provided by the Office upon request and payment of the necessary fee. The foregoing aspects and many of the attendant advantages of this invention will become more readily appreciated as the same become better understood by reference to the following detailed description, when taken in conjunction with the accompanying drawings, wherein:

FIGS. 1A-1F show Gene editing establishes VWF−/− hPSC lines. Summary of cell line cohort (FIG. 1A). Representative Sanger sequencing chromatograms of TOPO-cloned allelic DNA from (FIG. 1B) mutant 1 (SEQ ID NO: 5 and SEQ ID NO: 11) and (FIG. 1C) mutant 4 (SEQ ID NO: 7 and SEQ ID NO: 13), compared to isogenic control sequences representing portions of wild-type VWF (SEQ ID NO: 1) nucleotide sequence for mutant 1 (SEQ ID NO: 8), mutant 2 (SEQ ID NO: 9), and mutant 3 (SEQ ID NO: 10). TIDE analysis showing histogram of computationally inferred sequences based on Sanger sequencing of the gRNA locus (FIG. 1D). Histograms are shown in different colors for mutants (Mt) 1 (SEQ ID NO: 5 and SEQ ID NO: 11), 2 (SEQ ID NO: 6 and SEQ ID NO: 12), and 4 (SEQ ID NO: 7 and SEQ ID NO: 13). Mutant 3 sequence is the same as mutant 2. Brightfield images of control and VWF-1-colonies at passage 30 after genome editing (FIG. 1E). Representative images of teratoma tissue sections stained with hematoxylin and eosin, showing derivatives of endoderm (secretory epithelium), ectoderm (pigmented epithelium), and mesoderm (cartilage) (FIG. 1F). Descendants of the parental WTC-11 iPS cell line is shown. Scale bars, 200 μm.

FIGS. 2A-2I show VWF−/− hPSC differentiate efficiently into endothelial cells. (FIG. 2A) Schematic of hPSC-EC differentiation protocol. (FIG. 2B) Representative phase contrast images of control and VWF−/− iPS cells during differentiation into EC. (FIG. 2C) Representative immunoblot for VWF in hPSC-EC lysates of three VWF−/− clones, compared to HUVEC (HV), undifferentiated hPSC (SC), and three isogenic controls. (FIG. 2D) Quantification of immunoblot VWF protein levels normalized to b-actin (mean±s.e.m from n=8 independent experiments; ****, p<0.0001 by unpaired two-tailed t-test.) (FIGS. 2E-2F) Representative confocal optical sections showing co-localization of VWF with endothelial markers (FIG. 2E) CD144 and (FIG. 2F) CD31 by immunofluorescence in isogenic hPSC-EC on day 10 of differentiation. Arrows illustrate how line scans were drawn. (FIG. 2G) Averaged line scans showing fluorescence intensity of VWF and CD144 in control and VWF−/−(n=25 line scans per condition, pooled from 3 independent experiments). bpp, bytes per pixel. (FIG. 2H) EC differentiation efficiency from hPSC. Percentage of optical field covered by cells expressing CD144, analyzed from wide field immunofluorescence images of control and VWF−/− genotypes (mean±s.e.m., n=12 random fields pooled from 3 independent experiments). ns: not significant. (FIG. 2I) VWF multimeric size analysis by SDS-1.5% agarose gel electrophoresis in cell culture supernatants from isogenic control or VWF−/− hPSC-EC stimulated by 100 ng/ml PMA for 1 hour at 37° C., compared to human recombinant VWF (rVWF) expressed from HEK293 cells. Scale bars, 50 μm in (FIG. 2B) and 20 μm in (FIGS. 2E-2F).

FIGS. 3A-3G show VWF does not impact P-selectin or ANG2 expression in hPSC-EC. Representative immunofluorescence images (FIG. 3A) and quantification of P selectin and VWF (FIG. 3B) locations in control and VWF−/− hPSC-EC on day 12 of differentiation and HUVEC. Representative immunofluorescence images and (FIG. 3C) quantification of angiopoietin 2 (ANG2) (FIG. 3D) in control and VWF−/− hPSC-EC on day 12 of differentiation and HUVEC. n=8 fields of view per condition, pooled from two independent experiments. Error bars, s.e.m. Representative phase contrast images of tube formation in cultures of control or VWF−/− hPSC-EC (FIG. 3E). Scale bar, 500 μm. Tube length (FIG. 3F) and branch number (FIG. 3G) normalized to the mean value of control hPSC-EC. n=4 independent experiments. ns, not significant. Error bars, s.e.m.

FIGS. 4A-4H show Plasma increases VWF expression and maturity hallmarks in hPSC-EC. Timeline of plasma addition to derive hPSC-EC (FIG. 4A). Representative confocal immunofluorescence of EC populations derived with increasing plasma concentrations (FIG. 4B), with (FIG. 4C) zoomed images showing WPBs. Quantification of VWF intensity per cell normalized to the mean value of control without plasma treatment (n≥11 fields measured, pooled from three independent experiments) (FIG. 4D). Representative wide-field immunofluorescence images of VWF expression in isogenic control and VWF−/− hPSC-EC treated with 20% human plasma from day 7 to 12 of differentiation (FIG. 4E). Representative confocal immunofluorescence images (FIG. 4F), quantification of area circumscribed by CD144 (FIG. 4G), and line scan analysis of CD144 in cultures of hPSC-EC derived with increasing plasma concentrations (FIG. 4H). Arrows indicate thickened cell-cell junctions. For (FIG. 4G), n≥40 cells per condition (grey dots), pooled from three independent experiments. For (FIG. 4H), line scans (3.5 μm length) measuring fluorescence intensity (bpp, bytes per pixel) were drawn from one cell into its neighbor, through cell-cell junctional membranes. n=28 line scans per condition, pooled from three independent experiments. In (FIG. 4D), (FIG. 4G), and (FIG. 4H), *, p<0.05; ***, p<0.001; ****, p<0.0001 by ANOVA. Error bars, s.e.m.

FIGS. 5A-5I show DAPT treatment increases VWF expression. Fluorescence intensity of VWF per cell in hPSC-EC treated with ten different compounds: VEGF, FGF, 120 ng/ml; TGFb1, LPS, IFN, Noggin, 10 ng/ml; CHIR, SAG, 10 M; BMP4, 50 ng/ml, or DAPT, 1 μM, normalized to the mean value of untreated control hPSC-EC (FIG. 5A). The untreated media contained 20 ng/ml VEGF, 20 ng/ml FGF, and 1 μM CHIR. n≥8 fields of imaging field per condition. Quantification of VWF fluorescence intensity in cultures with 0 or 1 μM DAPT normalized to mean value of untreated control (mean±s.e.m. from n=3 independent experiments, each data point represents a single optical field; ****, p<0.0001 by unpaired two-tailed t-test) (FIG. 5B). Representative wide-field immunofluorescence images of hPSC-EC treated with increasing concentrations of DAPT. Scale bar: 30 μm (FIG. 5C). Representative immunoblot images (FIG. 5D), and quantification of VWF (FIG. 5E) or CD144 (FIG. 5F) (mean±s.e.m. from ≥5 independent experiments; **, p<0.01 by one-way ANOVA). Error bars, s.e.m. Representative immunofluorescence images (FIG. 5G), fluorescence intensity of VWF per cell (FIG. 5H), and representative immunoblot images of HUVEC treated with 0-30 μM of DAPT (FIG. 5I). Scale bar, 25 μm. Data were pooled from 3 independent experiments. *, p<0.05 by one-way ANOVA. Error bars, s.e.m.

FIGS. 6A-6D show DAPT treatment enables modeling of von Willebrand disease in hPSC-EC. Wide-field immunofluorescence images of CD144 (top) or CD31 (bottom) in flow channels containing hPSC-EC, with increasing zoom from left to right (FIG. 6A). Live wide-field immunofluorescence (FIG. 6B) and quantification of VWF (FIG. 6C) on isogenic control or VWF−/− hPSC-EC surfaces stimulated by 100 ng/mL PMA for 20 minutes at 370° C. and then followed by flow (2.5 dyn/cm² wall shear stress) for 5 minutes. Surface VWF was stained by FITC-labeled anti-VWF antibody without cell permeabilization. VWF−/− hPSC-EC was used as a negative control for non-specific bound fluorescence at the cell surface. Quantification shows total VWF area (over threshold) attached to the EC surface, per imaging field, excluding strings. Each data point was normalized to the mean value of VWF area on VWF−/− hPSC-EC. n≥30 images/condition pooled from three independent experiments. ****, p<0.0001 by student t test. Error bars, s.e.m. (FIG. 6D) Images similar to (FIG. 6B), showing formation of VWF strings at the surface of control cells.

FIGS. 7A-7E show VWF is dispensable for hPSC-EC differentiation. Representative Sanger chromatograms of TOPO cloned allelic DNA from mutant 2 (SEQ ID NO: 6 and SEQ ID NO: 12), compared to control (SEQ ID NO: 9 and SEQ ID NO: 1). Allele sequences are shown below. Asterisk marks start of the gRNA sequence (FIG. 7A). Representative wide-field immunofluorescence images of HUVEC and hPSC-EC, showing WPB morphology (FIG. 7B). Representative electron microscopy images of WPBs in HUVEC and WPB-like structures in hPSC-EC. Yellow arrows: WPBs or WPB-like structures (FIG. 7C). Representative confocal immunofluorescence images of VWF−/− hPSC-EC on day 13 of cell differentiation, 48 hours after transfection with either empty vector (pcDNA3.1) or vector encoding a full length human VWF (SEQ ID NO: 14) (FIG. 7D). Confocal optical sections showing proximal tubules (LTL+) and EC (CD31+) in kidney organoid cultures differentiated from control or VWF−/− hPSC (FIG. 7E).

FIGS. 8A-8C show treatment with blood plasma or FBS increases VWF levels in hPSC-EC. Representative immunoblot images of VWF expression level in hPSC-EC treated with 0%-80% human blood plasma starting on day 7 of cell differentiation (FIG. 8A). Representative wide-field immunofluorescence images (FIG. 8B) and quantification of hPSC-EC (FIG. 8C) treated with additional 5, 10, or 20% of FBS in cell culture medium starting on day 7 of differentiation. n≥12 imaging fields per condition, pooled from 3 independent experiments. *, p<0.05, **, p<0.01 by one-way ANOVA. Error bars, s.e.m. Scale bar, 10 μm.

FIGS. 9A-9C show DAPT treatment increases VWF level in hPSC-EC but does not affect CD144 marker for EC. Quantification t experiments. **, p<0.01 by one-way ANOVA). Mixed effect model was used to calculate statistics (FIG. 9A). Representative immunoblot for full-length Notch1 and cleaved Notch1 intracellular domain in hPSC-EC (FIG. 9B). Representative wide-field immunofluorescence images for hPSC-EC treated with 0 or 3 μM DAPT. Scale bar, 5 μm (FIG. 9C).

FIGS. 10A-10B show VWF forms strings on the EC surface upon PMA stimulation. Representative live wide-field immunofluorescence images for VWF on the EC surface under flow in a microfluidic device in the presence of 100 nM PMA for HUVEC (FIG. 10A) on day 7 after cell seeding and hPSC-EC (FIG. 10B) on day 12 of cell differentiation.

### DETAILED DESCRIPTION

VWF is a multimeric glycoprotein found in blood plasma, the subendothelial matrix, and storage granules in endothelial cells, the Weibel-Palade bodies (WPBs), and platelets (α-granules). Although some unique functional features of VWF have recently been proposed, the protein is best recognized for its role in the hemostatic process it mediates platelet adhesion and aggregation at sites of vascular damage and transports coagulation factor VIII (FVIII) in the bloodstream. Patients who lack VWF exhibit a severe hemorrhagic phenotype caused by poor platelet-rich thrombi development and a subsequent FVIII deficiency that impairs fibrin network production. Functional and/or quantitative deficits of VWF are known as von Willebrand disease (VWD), a condition that affects 0.01% to 1% of the population. Quantitative deficiencies of VWF correlate with changes in biosynthesis, secretion, and/or clearance of the protein.

Progenitor cells and their differentiated progeny can be used in cellular assays, drug screening, and toxicity assays. Progenitor cells and their differentiated progeny also show promise for cell-based therapies, such as in regenerative medicine for the treatment of damaged tissue. Human embryonic stem cells (hESC) can be propagated and expanded indefinitely in vitro, providing an inexhaustible and potentially donor-free source of cells for human therapy. EC derived from human pluripotent stem cells (hPSC-EC) have potential advantages in reproducibility, self-renewal, gene editing, and patient-specific disease modeling. As hPSCs are pluripotent, they have the potential to differentiate into sophisticated structures, and may provide a source of immunocompatible replacement tissue. Coupled with microfluidics, hPSC-ECs have been used to establish advanced microphysiological models of vasculature. The maturation state of hPSC-ECs remains unclear, however, and a well-controlled, gene edited model of VWF deficiency in hPSC-EC has not yet been developed. The pathways that regulate VWF expression levels in EC have not been characterized in detail, which impedes the usage of hPSC-EC as a cellular model to study VWF function, related diseases, and potential therapeutics.

The present disclosure provides important insights into the role and control of VWF expression in human EC for disease modeling. By knocking out VWF, the inventors have developed VWF-deficient hPSC-EC models and demonstrated that hPSC differentiation, angiopoietin expression, and P selectin are not dependent on VWF. It was also observed that WPBs are rounder and VWF expression level is lower in hPSC-EC, compared to primary EC (HUVEC). These differences likely reflect challenges in the maturation of EC derived via hPSC differentiation, or differences between specific subtypes of EC derived using any given protocol. This model offers a platform to study VWF-related EC function and type 3 Von Willebrand Factor disease (VWF) which features loss of VWF. As these cell lines are gene edited and immortal, they are a resource for future applications relevant to VWF, for instance, the present disclosure contemplates coupling these with high throughput methods to identify novel interactors of VWF, potential therapeutic agents, and/or naturally occurring anti-VWF antibodies (inhibitors).

Furthermore, hPSC-ECs comprising intact VWF were observed to express and secrete VWF multimers, but not efficiently form VWF strings on the cell surface under standard culture conditions when stimulated. Treating hPSC-EC culture medium with a small molecule, DAPT, upregulates VWF expression, enabling VWF string formation at the EC surface. As DAPT also increases VWF in adult (primary) EC cultures, this is a general effect that is not limited to the hPSC system or differentiation per se. DAPT inhibits Notch signaling, which may be responsible for the increase of VWF expression. Alternatively, DAPT may inhibit the proteasome and thus increase VWF levels by blocking its degradation. The effects of DAPT on VWF expression in EC suggest a new potential clinical application for DAPT in treating forms of VWD in which VWF is expressed at low quantity levels. Alternatively, these data also suggest that thrombosis may be a possible adverse event that could result from treatment with DAPT at high doses.

Interestingly, exposure to human blood plasma also substantially enhanced VWF expression levels in hPSC-ECs harboring intact VWF. Blood is part of the natural growth environment of EC, and blood plasma contains many growth factors, which could affect VWF expression. The screening experiments of the present disclosure suggest that plasma's effect is unlikely to be mediated by FGF, TGF, or VEGF. Compared to the spherical WPBs in untreated hPSC-EC, WPBs in plasma-treated hPSC-EC adopt a more rod-shaped morphology, which more closely resembles WPBs in primary EC. Beyond its effects on VWF, plasma also increased the size of hPSC-EC and improved the morphology of their cell-cell adherens junctions. Therefore, blood plasma may be essential to EC growth and hPSC-EC maturation. Platelets are also thought to play an important role in EC function during vascular maturation and therefore treatment with platelet-rich plasma potentially affects VWF expression and hPSC-EC function. Moreover, the effects of plasma may not be limited just to ECs but also may broadly extend to other lineages derived from hPSCs.

The present disclosure provides hPSC-ECs and cellular models comprising hPSC-ECs that have a capacity to serve as a renewable, editable, and unlimited endothelial cell source. The present disclosure also provides VWF-deficient hPSC-ECs as a novel tool to study VWF function in human disease states. Further, the present disclosure establishes exposure to human blood plasma as a new methodology to improve the maturation state of hPSC-ECs. DAPT shows great potential to enhance VWF expression in ECs and may serve as a possible therapeutic for treating bleeding disorders.

To facilitate an understanding of the present disclosure, a number of terms and phrases are defined below.

The term “cell line” refers to a mortal or immortal population of cells that is capable of propagation and expansion in vitro.

The term “clonal” refers to a population of cells obtained from the expansion of a single cell into a population of cells all derived from that original single cell and not containing other cells.

The term “differentiated cells” when used in reference to cells made by methods of this disclosure from pluripotent stem cells refer to cells having reduced potential to differentiate when compared to the parent pluripotent stem cells. The differentiated cells of this invention comprise cells that could differentiate further (i.e., they may not be terminally differentiated).

The term “organoid” refers to cultured cells that are aggregated to create a three-dimensional structure with tissue-like cell density such as occurs in the culture of some cells over a layer of agar, cultured as teratomas in an animal, otherwise grown in a three-dimensional culture system but wherein said aggregated cells contain cells of different cell lineages, such as, by way of nonlimiting examples, the combination of epidermal keratinocytes and dermal fibroblasts, or the combination of parenchymal cells with their corresponding tissue stroma, or epithelial cells with mesenchymal cells.

The term “pluripotent stem cells” refers to animal cells capable of differentiating into more than one differentiated cell type. Such cells include hES cells, blastomere/morula cells and their derived hED cells, hiPS cells, hEG cells, hEC cells, and adult-derived cells including mesenchymal stem cells, neuronal stem cells, and bone marrow-derived stem cells. Primordial stem cells may be from non-human animals. Primordial stem cells may be genetically modified or not genetically modified. Genetically modified cells may include markers such as fluorescent proteins to facilitate their identification in vitro or in vivo.

The term “induced pluripotent stem cell” encompasses pluripotent cells that, like embryonic stem (ES) cells, can be cultured over a long period of time while maintaining the ability to differentiate into all types of cells in an organism, but that, unlike ES cells (which are derived from the inner cell mass of blastocysts), are derived from differentiated somatic cells, that is, cells that had a narrower, more defined potential and that in the absence of experimental manipulation could not give rise to all types of cells in the organism. iPS cells have an hESC-like morphology, growing as flat colonies with large nucleo-cytoplasmic ratios, defined borders and prominent nuclei. In addition, iPS cells express one or more key pluripotency markers known by one of ordinary skill in the art, including but not limited to alkaline phosphatase, SSEA3, SSEA4, Sox2, Oct3/4, Nanog, TRA160, TRA181, TDGF 1, Dnmt3b, FoxD3, GDF3, Cyp26al, TERT, and zfp42. In addition, the iPS cells are capable of forming teratomas. In addition, they are capable of forming or contributing to ectoderm, mesoderm, or endoderm tissues in a living organism.

Genes may be introduced into the cells disclosed herein or cells derived therefrom for a variety of purposes, e.g. to replace genes having a loss of function mutation, provide marker genes, etc. Alternatively, vectors are introduced that express guide RNAs, CRISPR/CAS9 system, antisense mRNA or ribozymes, thereby blocking expression of an undesired gene. Other methods of gene therapy are the introduction of drug resistance genes to enable normal progenitor cells to have an advantage and be subject to selective pressure, for example the multiple drug resistance gene (MDR), or anti-apoptosis genes, such as bcl-2. Various techniques known in the art may be used to introduce nucleic acids into the target cells, e.g. electroporation, calcium precipitated DNA, fusion, transfection, lipofection, infection and the like, as discussed above. The particular manner in which the DNA is introduced is not critical to the practice of the invention.

In addition to various uses as an in vitro cultured cells, the hPSC-ECs of the present disclosure may also be utilized to generate cell lines, tissues, teratomas, and organoids. Further, the hPSC-ECs disclosed herein may be tested in a suitable animal model. At one level, cells are assessed for their ability to survive and maintain their phenotype in vivo. Cell compositions are administered to immunodeficient animals (such as nude mice, or animals rendered immunodeficient chemically or by irradiation). Tissues are harvested after a period of regrowth, and assessed as to whether the administered cells or progeny thereof are still present and may be phenotyped for response to a treatment of interest.

As used herein, the term “individual,” “subject,” or “patient,” used interchangeably, refers to any animal, including mammals, preferably mice, rats, other rodents, rabbits, dogs, cats, swine, cattle, sheep, horses, or primates, and most preferably humans.

The terms “treatment,” “treating,” “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

One letter codes for amino acids are used herein. For example, alanine is A, arginine is R, asparagine is N, aspartic acid is D, asparagine or aspartic acid is B, cysteine is C, glutamic acid is E, glutamine is Q, glutamine or glutamic acid is Z, glycine is G, histidine is H, isoleucine is I, leucine is L, lysine is K, methionine is M, phenylalanine is F, proline is P, serine is S, threonine is T, tryptophan is W, tyrosine is Y, valine is V.

As used herein, the term “nucleic acid” refers to a polymer of nucleotide monomer units or “residues.” The nucleotide monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art. Modifications to the nucleic acid monomers, or residues, encompass any chemical change in the structure of the nucleic acid monomer, or residue, that results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5-carboxycytosine b-glucosyl-5-hydroxy-methylcytosine, 8-oxoguanine, 2-amino-adenosine, 2-amino-deoxyadenosine, 2-thiothymidine, pyrrolo-pyrimidine, 2-thiocytidine, or an abasic lesion. An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA.

Reference to sequence identity addresses the degree of similarity of two polymeric sequences, such as nucleic acid or protein sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain,” as used herein, is a protein fragment comprising an active or inactive cleavage domain of Cas9 and/or the gRNA binding domain of Cas9. A “Cas9 protein” is a full length Cas9 protein. A Cas9 nuclease is also referred to sometimes as a casnl nuclease or a CRISPR (Clustered Regularly Interspaced Short Palindromic Repeat-associated nuclease). CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 domain. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species.

As used herein, the terms “upstream” and “downstream” are terms of relativity that define the linear position of at least two elements located in a nucleic acid molecule (whether single or double-stranded) that is orientated in a 5′-to-3′ direction. In particular, a first element is upstream of a second element in a nucleic acid molecule where the first element is positioned somewhere that is 5′ to the second element. Conversely, a first element is downstream of a second element in a nucleic acid molecule where the first element is positioned somewhere that is 3′ to the second element.

As used herein, the term “guide RNA” is a particular type of guide nucleic acid which is mostly commonly associated with a Cas protein of a CRISPR-Cas9 and which associates with Cas9, directing the Cas9 protein to a specific sequence in a DNA molecule that includes complementarity to protospacer sequence of the guide RNA. However, this term also embraces the equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas systems), C2cl (a type V CRISPR-Cas system), C2c2 (a type VI CRISPR-Cas system) and C2c3 (a type V CRISPR-Cas system). As used herein, the “guide RNA” may also be referred to as a “traditional guide RNA” to contrast it with the modified forms of guide RNA termed “prime editing guide RNAs” (or “pegRNAs”) which have been invented for the prime editing methods and composition disclosed herein. As used herein, the term “organs” refers to a group of tissues in a living organism that have been adapted to perform a specific function.

As used herein, the term “protein” refers to any of various naturally occurring macromolecules comprising one or more polypeptide chains (also referred to herein as protein “subunits”). A “polypeptide” is a polymer of amino-acid residues joined by peptide bonds. A protein may also comprise non-peptidic components, such as carbohydrate groups, which may be added to a protein by the cell in which the protein is produced. Proteins contain the elements carbon, hydrogen, nitrogen, oxygen, usually sulfur, and occasionally other elements (such as phosphorus or iron), and include many essential biological compounds (such as enzymes, hormones, or antibodies).

As used herein, the term “small molecule” refers to a low molecular weight (<2000 daltons) organic compound that may help regulate a biological process, with a size on the order of 1 nm. Most drugs are small molecules.

As used herein, the term “therapeutic agent” refers to a substance capable of producing an improvement and/or a curative effect in a disease state.

As used herein, the term “isolated” in regard to cells, refers to a cell that is removed from its natural environment.

As used herein, the term “tissue” refers to an aggregate of similar cells and cell products forming a definite kind of structural material with a specific function in a multicellular organism.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full-length coding sequence or by any portion of the coding sequence so long as the desired activities or functional properties (e.g., enzymatic activity, ligand binding, signal transduction, immunogenicity, etc.) of the full-length polypeptide or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns can contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In accordance with the foregoing, the present disclosure provides methods and compositions useful for generating cellular models from human pluripotent stem cells and methods for application of these cellular models, in combination with Organ-on-chip and compound/therapeutic agent screening techniques, for fundamental studies of VWF function, accurate detection of VWF expression, understanding the effect of VWF expression on the expression of related genes and protein, improvement of endothelial cell differentiation, binding of platelet (GPIbalpha), binding of factor VIII, as well as for developing patient-specific therapeutics for hemostasis and thrombosis. Specifically, the disclosed methods and related compositions can be integrated into methods of medical intervention. Various aspects of the disclosure are addressed herein.

Methods are provided for the generation of an in vitro cellular model of VWF disease comprising VWF disease-relevant endothelial cells. The VWF disease relevant Endothelial cells (ECs) are differentiated from human pluripotent stem cells (hPSCs). In an embodiment, the human pluripotent stem cells (hPSCs) are treated with an agent effective in suppressing or deleting at least one gene sequence or a portion thereof, encoding a protein and/or a subunit thereof, where the protein and/or subunit thereof is associated with VWF-linked secretion. In some embodiments, the human pluripotent stem cells (hPSCs) are treated with the agent prior to differentiation. In some embodiments, the human pluripotent stem cells (hPSCs) are treated with the agent after differentiation. In an embodiment, the suppressing or deleting of at least one gene sequence or a portion thereof encoding a protein and/or a subunit thereof changes an amount of VWF linked protein secretion. In an embodiment, the protein of interest associated with VWF-linked secretion includes, without limitation, the von Willebrand Factor protein (VWF). In some embodiments, the agent is effective in disrupting the VWF gene to produce a pluripotent stem cell-derived endothelial cell with knock out mutation of VWF. In some embodiments, the agent effective in disrupting the VWF gene comprises a CRISPR/Cas9 genome editing system comprising nucleotide sequences encoding CRISPR-Cas guide RNAs. In some embodiments, the guide RNAs hybridize with a target sequence in exon 1 or in exon 2 of the VWF gene.

In an embodiment, the present disclosure provides for a method for the generation of endothelial cells differentiated from human pluripotent stem cells. In an embodiment, the method provides for the generation of endothelial cells with intact VWF-linked protein secretion. In an embodiment, the method provides for the generation of endothelial cells expressing VWF protein. In some embodiments, the method provides for the generation of endothelial cells with disrupted VWF-linked protein secretion. In some embodiments, the method provides for the generation of endothelial cells lacking VWF protein.

In some embodiments of the present disclosure, the hPSC-ECs disclosed herein are capable of forming a cell line, a teratoma, a tissue, or an organoid.

In some embodiments, the present disclosure pertains to a high throughput method for identifying a therapeutic agent for treating bleeding disorders. For example, according to this aspect of the disclosure, a candidate compound/agent may be contacted with a cell or an organoid as described herein, and any change to the cell or in activity of the cell may be monitored. For high-throughput purposes, said organoids are cultured in multiwell plates such as, for example, 96 well plates or 384 well plates or in microfluidic devices such as organ-on-chip devices. Libraries of molecules/agents are used to identify an agent that has a therapeutic effect on the cell or organoid.

Thus, in an embodiment, the method comprises contacting the hPSC-ECs of the present disclosure with an effective amount of a plurality of candidate agents and identifying an agent capable of modulating the expression of a protein or a subunit thereof associated with VWF-linked protein secretion, enhancing the expression of VWF protein or a subunit thereof, restoring the expression of VWF, restoring VWF-linked protein secretion, and/or restoring VWF string formation, as the therapeutic agent effective for treating bleeding disorders. The therapeutic agents effective in treating bleeding disorders include but are not limited to genetic agents, small molecules, and components of cell culture environment.

In some embodiments, the genetic agent comprises a vector comprising a polynucleotide encoding VWF protein, a subunit of VWF, or a derivative thereof, a polynucleotide encoding a protein linked/associated with VWF-linked protein secretion, a subunit, or a derivative thereof, or a polynucleotide targeting a regulator or suppressor of a protein associated with VWF-linked protein secretion. In some embodiments, the genetic agent is effective in enhancing the expression of VWF protein, restoring the expression of VWF, restoring VWF-linked protein secretion, and/or restoring VWF string formation.

In some embodiments, the small molecule modulates a protein linked/associated with VWF-linked protein secretion, a subunit, or a derivative thereof. In some embodiments, the small molecule is an agonist of VWF expression. In an embodiment, the small molecule comprises DAPT. In some embodiments, the small molecule is effective in enhancing the expression of VWF protein, restoring the expression of VWF, restoring VWF-linked protein secretion, and/or restoring VWF string formation.

In some embodiments, the component of cell culture environment modulates a protein linked/associated with VWF-linked protein secretion, a subunit, or a derivative thereof. In some embodiments, the component of cell culture environment is effective in enhancing the expression of VWF protein, restoring the expression of VWF, restoring VWF linked protein secretion, and/or restoring VWF string formation. In some embodiments, the component of cell culture environment is blood plasma.

In some embodiments the method comprises providing a panel of hPSC-ECs of the present disclosure, where the panel includes the VWF disease-relevant endothelial cells and control endothelial cells comprising intact VWF-linked secretion. In some embodiments a panel of such ECs are contacted with a plurality of candidate agents, or a plurality of doses of a therapeutic agent.

As used herein, the term “genetic agent” refers to polynucleotides and analogs thereof, which agents are tested in the high throughput methods of the disclosure by addition of the genetic agent to a cell. The introduction of the genetic agent results in an alteration of the total genetic composition of the cell. Genetic agents such as DNA can result in an experimentally introduced change in the genome of a cell, generally through the integration of the sequence into a chromosome. Genetic changes can also be transient, where the exogenous sequence is not integrated but is maintained as an episomal agent. Genetic agents, such as antisense oligonucleotides, can also affect the expression of proteins without changing the cell's genotype, by interfering with the transcription or translation of mRNA. The effect of a genetic agent is to increase or decrease expression of one or more gene products in the cell.

Introduction of an expression vector encoding a polypeptide can be used to express the encoded product in cells lacking the sequence, or to over-express the product. Various promoters can be used that are constitutive or subject to external regulation, where in the latter situation, one can turn on or off the transcription of a gene. These coding sequences may include full-length cDNA or genomic clones, fragments derived therefrom, or chimeras that combine a naturally occurring sequence with functional or structural domains of other coding sequences. Alternatively, the introduced sequence may encode an anti-sense sequence; be an anti-sense oligonucleotide; RNAi, encode a dominant negative mutation, or dominant or constitutively active mutations of native sequences; altered regulatory sequences, etc.

In the high throughput methods for identifying genetic agents, polynucleotides are added to one or more of the cells in order to alter the genetic composition of the one or more cell. The output parameters are monitored to determine whether there is a change in phenotype. In this way, genetic sequences are identified that encode or affect expression of proteins in pathways of interest. The results can be entered into a data processor to provide a screening results dataset. Algorithms are used for the comparison and analysis of screening results obtained under different conditions.

In the high throughput methods for identifying small molecules, the effect of adding a candidate small molecule to cells in culture is tested with a panel of cells, where panels of cells may vary in genotype, or in the dose of agent that is provided, etc., where usually at least one control is included, for example a negative control and a positive control. In certain embodiments, libraries of compounds of small molecules are screened using the methods described herein.

In the high throughput methods for identifying a component of the cell culture the effect of altering the cell culture environment is assessed by adding or depleting a candidate agent in culture of cells and/or varying culture environments. Culture of cells is typically performed in a sterile environment, for example, at 37° C. in an incubator containing a humidified 92-95% air/5-8% CO₂ atmosphere. Cell culture may be carried out in nutrient mixtures containing undefined biological fluids such as fetal calf serum, or media which is fully defined and serum free. The effect of the candidate agent is assessed by monitoring multiple output parameters, including morphological, functional, and genetic changes.

Output parameters are quantifiable components of cells, particularly components that can be accurately measured, desirably in a high throughput system. A parameter can also be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value, or the variance, etc. Variability is expected and a range of values for each of the set of test parameters will be obtained using standard statistical methods with a common statistical method used to provide single values.

Exemplary output parameters include but are not limited to assessing VWF transcript expression, VWF protein expression, VWF localization patterns, formation of intracellular Weibel-Palade bodies containing VWF with rod-like morphology, and/or secretion of VWF multimers.

Preferred therapeutic agent does not include additional components, such as preservatives, that may have a significant effect on the overall formulation. Thus, preferred formulations of therapeutic agent consist essentially of a biologically active compound and a physiologically acceptable carrier, e.g. water, ethanol, DMSO, etc.

Toxicology screens work in a similar way to methods for identifying a therapeutic agent (as described above) but they test for the toxic effects of the agent and not therapeutic effects. Toxicity assays may be in vitro assays using cell lines, an organoid or part thereof, or a cell derived from a cell line, or an organoid disclosed herein. It is anticipated that toxicity results obtained with organoids more closely resemble results obtained in patients. For example, according to this aspect of the disclosure, a candidate compound may be contacted with a cell, or an organoid as described herein, and any change to the cells or in activity of the cells may be monitored. For high-throughput purposes, said cells or organoids are cultured in multiwell plates such as, for example, 96 well plates or 384 well plates or in microfluidic devices such as organ-on-chip device. Libraries of molecules are used to identify a molecule that affects said organoids.

The present disclosure also pertains to cell lines, teratomas, tissues, and/or an organoid obtained from the hPSC-ECs disclosed herein. In certain embodiments, the present disclosure provides an organoid in which at least 40%, at least 50%, at least 60%, at least 70%, at least 80%, at least 90% or at least 99% of the cells express endothelial cell markers.

The present disclosure provides for the use of an organoid or cells derived from said organoid in drug screening, (drug) target validation, (drug) target discovery, toxicology and toxicology screens, personalized medicine, regenerative medicine and/or as ex vivo cell/organ models, such as disease models of VWF disease. The organoids of the present disclosure provide a genetically stable platform which faithfully represents the in vivo situation. In some embodiments, the organoids of the present disclosure comprise all differentiated cell types that are present in the corresponding in vivo situation. In other embodiments, the organoids of the present disclosure may be further differentiated to provide all differentiated cell types that are present in vivo. Thus, the organoids of the present disclosure can be used to gain mechanistic insight into VWF disease and therapeutics, to carry out in vitro drug screening, to evaluate potential therapeutics, to identify possible targets (e.g. proteins) for future novel (drug) therapy development and/or to explore gene repair coupled with cell-replacement therapy.

The organoids of the present disclosure can be frozen and thawed and put into culture without losing their genetic integrity or phenotypic characteristics and without loss of proliferative capacity. Thus, the organoids can be easily stored and transported. In some embodiments, the present disclosure provides a frozen organoid.

In certain embodiments, the present disclosure further provides the use of a cell line, a tissue, a teratoma, an organoid of the disclosure, or a cell derived from said cell line, tissue, teratoma, or organoid, in a drug discovery screen; toxicity assay; pharmacokinetics; diagnostics; establishing cell lineages; research of differentiation pathways; gene expression studies including recombinant gene expression or etiology of bleeding disorders.

The present disclosure also pertains to microfluidic devices comprising the hPSC-ECs, or cells obtained from the cell lines, teratomas, tissues, and/or organoids disclosed herein. In some embodiments, the microfluidic device comprises an organ-on-chip configured to represent VWF disease.

The present t disclosure also contemplates assessing toxicity and/or pharmacokinetics of the identified therapeutic agent using the cells, cell lines, teratomas, organoids, and devices disclosed herein.

Also provided herein is a method of treating a bleeding disorder in a subject in need thereof. In some embodiments, the method comprises administering to the subject an effective amount of a therapeutic agent identified by the methods disclosed herein. In some embodiments, the method is effective in restoring VWF string formation. Accordingly, included within the scope of the disclosure are methods of treatment of a human or non-human animal patient. The term “animal” here denotes all mammalian animals. The subject may be at any stage of development, including embryonic and fetal stages. For example, the subject may be an adult, or the therapy may be for pediatric use (e.g. newborn, child or adolescent). The method of treatment encompasses the administration of the therapeutic agent identified by the methods disclosed herein to a subject through any appropriate means. The term “administration” as used herein refers to well-recognized forms of administration, such as, for example, oral, intravenous, intramuscular, subcutaneous, intra-atrial, intra-articular, parenteral, intranasal, intrapulmonary, transdermal, intrapleural, intrathecal, and oral routes of administration. A therapeutic agent may be administered to a subject in a single bolus delivery, via continuous delivery (e.g., continuous transdermal delivery) over an extended time period, or in a repeated administration protocol (e.g., on an hourly, daily, or weekly basis).

For convenience, the cells, cell lines, teratomas, tissues, organoids, and devices disclosed herein may be provided in kits. The kits could include the cells to be used, which may be frozen, refrigerated or treated in some other manner to maintain viability, reagents for measuring the parameters, and software for preparing the screening results. The software will receive the results and perform analysis and can include reference data. The software can also normalize the results with the results from a control culture. The composition may optionally be packaged in a suitable container with written instructions for a desired purpose, such as screening methods, and the like.

For further elaboration of general techniques useful in the practice of the methods and systems disclosed herein, the practitioner can refer to standard textbooks and reviews in cell biology, tissue culture, embryology, and cardiophysiology. With respect to tissue culture and embryonic stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998).

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen. Sigma-Aldrich, and Clon Tech.

The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### Example 1

### Cell Lines and Genome Editing

Cell lines used to generate VWF/hPSC lines included H9/WA09 human embryonic stem (ES) cells (WiCell) and WTC11 human induced pluripotent stem (iPS) cells (Coriell GM25256) lines. Cell lines were maintained in mTeSR1 (StemCell Technologies) with Penicillin-Streptomycin (Gibco) on reduced growth factor GelTrex (Gibco) coated cell culture plate. To create VWF^{-/-} hPSC lines, the CRISPR (clustered regularly interspaced short palindromic repeats) method was applied by transfecting plasmids encoding guide RNA and GFP-tagged Staphylococcus aureus Cas9 into hPSC, sorting the transfected cells for GFP, and picking the resultant colonies as described. Guide RNA (gRNA) sequences AACTCGCGGCAGGTCATCCACGG (SEQ ID NO: 2) and GTCAATGGTACCGTGACACAGGG (SEQ ID NO: 3), targeting exons 2 and 3 in the VWF gene (SEQ ID NO: 1), were used separately to knock out VWF in WTC11 or H9/WA09 cell lines, respectively. VWF knockout clones (Mutant 1 (SEQ ID NO: 5 and SEQ ID NO: 11), Mutant 2/3 (SEQ ID NO: 6 and SEQ ID NO: 12), and Mutant 4 (SEQ ID NO: 7 and SEQ ID NO: 13) were confirmed by Sanger sequencing chromatograms of TOPO-cloned allelic DNA compared to isogenic control sequences (SEQ ID NO: 1 and corresponding portions of SEQ ID NO: 1 as shown in SEQ ID NOs: 8-10) and TIDE (tracking of indels by Decomposition) analysis. hPSC colonies showing no edits at the target were expanded as isogenic controls.

### Example 2

Differentiation of hPSC Lines into Endothelial Cells

hPSC were differentiated into endothelial cells (hPSC-EC) based on a previously established differentiation protocol. Palpant, N.J. et al. Generating high-purity cardiac and endothelial derivatives from patterned mesoderm using human pluripotent stem cells. Nat Protoc 12, 15-31 (2017). The hPSC were maintained feeder-free on 1% Geltrex reduced growth factor basement membrane matrix (Thermo Fisher Scientific) plate in mTeSR1 medium (StemCell Technologies) and dissociated with Accutase (StemCell Technologies). Total 50,000 cells from each cell line were plated per well of a 24-well plate pre-coated with 1% Geltrex in mTeSR1 supplemented with 10 μM Rock inhibitor Y27632 (BD Biosciences) and 1 μM Chir-99021 (Tocris). For each well, the media was replaced with 500 μL Advanced RPMI 1640 media (Thermo Fisher Scientific)+1× B-27 supplement minus insulin (Thermo Fisher Scientific)+1:60 diluted Matrigel Growth Factor Reduced Basement Membrane Matrix (Corning)+50 ng/ml Activin A (R&D Systems) at 24 hours after cell seeding. On day 1, the cells were fed with 500 μL RPMI 1640 media+1× B-27 supplement minus insulin+40 ng/mL human bone morphogenetic protein 4 (BMP4, R&D Systems)+1 μM Chir99021 at 61 hours after cell seeding. On day 2, the cells were fed with 1000 μL StemPro-34 SFM (Thermo Fisher Scientific)+2 mM L-glutamine (Thermo Fisher Scientific)+1:80 diluted 1-thioglycerol (Sigma Aldrich)+50 μg/mL ascorbic acid (Sigma Aldrich)+10 ng/mL BMP4+5 ng/mL human fibroblast growth factor-basic (FGF, PeproTech)+200 ng/mL recombinant human VEGF₁₆₅ (VEGF, PeproTech) at 85 hours after cell seeding for a 72-hour incubation. On day 5, cells were passaged using Versene (Thermo Fisher Scientific) and 0.25% trypsin (Thermo Fisher Scientific) and seeded at density of 10⁴ cells per cm² onto a plate pre-coated with 0.1% gelatin (StemCell Technologies) in EGM endothelial cell growth medium (Lonza)+20 ng/mL VEGF+20 ng/mL FGF+1 μM Chir99021. Cells were then fed every other day with the same medium as the one on day 5. Definitive endothelial cells could be observed on day 10-14.

### Example 3

### Teratoma Formation

hPSC at the density of 300,000/well were seeded onto each well of a 6-well plate from isogenic control or VWF^{-/-} hPSC and allowed to reach confluent over six days of maintenance culture. Three wells were subsequently dissociated, pelleted, and resuspended in 250 μL of 1:1 mixture of Matrigel: DMEM/F12 medium (Corning and Thermo Fisher Scientific) maintained at 4 degrees Celsius. This mixture, containing ˜1×10⁷ hPSC, was rapidly injected beneath the neck scruff of two NOD-SCID mice (NOD.CB17-Prkdescid/J, Jackson Labs) using a 22-gauge needle. Tumors were palpable by 8 weeks post-injection and harvested at 12 weeks, fixed (60% methanol, 30% chloroform, 10% acetic acid, all from Sigma), and processed for histological analysis. All experiments with animals were performed under the auspices of the University of Washington IACUC under an approved protocol number 4375-01.

### Example 4

Endothelial Cell Differentiation from VWF^{-/-} hPSC in Kidney Organoid

Kidney organoids were prepared with 25 ng/ml supplementary VEGF as described. Briefly, to induce kidney organoid differentiation, hPSCs were dissociated and seeded onto 1% Growth factor reduced Matrigel (Corning)-coated 24-well plates at a density of 1500 cells/well. The following morning (d1), the cells were sandwiched with 1.5% Matrigel in mTeSR1, and fed with mTeSR1 the next day (d2), resulting in the formation of epiblast spheroids with hollow lumens. The following evening, organoids were treated with 12 μM Chir99021 in Advanced RPMI 1640 supplemented with GlutaMAX and Penicillin-streptomycin (all from Thermo Fisher Scientific) for 36 hours. Media was replaced with Advanced RPMI 1640 supplemented with GlutaMAX and Penicillin-streptomycin plus 1× B-27 supplement minus insulin (Thermo Fisher Scientific) and 25 ng/ml VEGF₁₆₅ (PeproTech), replaced two days later, then replaced every third day for two weeks until organoids formed enriched with vasculature.

### Example 5

### Immunofluorescence Staining

Cells were fixed with 4% paraformaldehyde in 1×PBS for 15 minutes and subsequently washed three times with 1×PBS. For immunofluorescence, fixed samples were blocked with 5% donkey serum plus 0.1% Triton X-100 in 1×PBS, incubated overnight in 3% bovine serum albumin (BSA) (Sigma Aldrich)+1×PBS with primary antibodies, washed, incubated with secondary antibodies, washed and imaged. Primary antibodies included antibodies for VWF (Agilent Technologies; A008202), β-Actin (Cell Signaling Technology; 4970); DAPI (Cayman Chemical Company; 14285), Angiopoietin 2 (R&D Systems; AF623), CD31 (BD Biosciences; 555444), CD144 (BD Biosciences; 555661), and Notch1 (Cell Signaling Technology; 3608S). Fluorescence labeled antibodies included anti-rabbit IgG secondary antibody Alexa Fluor 488 (Thermo Fisher Scientific; A32731), anti-mouse IgG secondary antibody Alexa Fluor 555 (Thermo Fisher Scientific; A21422), and FITC anti-VWF antibody (Abcam; ab8822). To compare different experimental conditions, images were collected at identical microscopy setup using wide field or confocal fluorescence microscope. Image analysis was performed using Fiji software.

### Example 6

### Western Blot

Whole cells lysate was extracted using 1× RIPA lysis and extraction buffer (Thermo Fisher Scientific) supplemented with 1× protease inhibitor cocktail (Sigma Aldrich) and 1 μL Benzonase nuclease (Sigma Aldrich) per sample. Each sample was supplied with 100 mM DTT (Sigma Aldrich) and 1× Laemmli sample buffer (Bio-Rad) and heated at 95° C. for 5 minutes. The primary antibodies above were added to samples and followed by anti-rabbit or anti-mouse IgG secondary antibody conjugated with HRP (Thermo Fisher Scientific; 31460 or 31430). Immunoblots were imaged in the presence of Pierce ECL Western Blotting substrate (Thermo Fisher Scientific) and quantified using Fiji software.

### Example 7

### Electron Microcopy Imaging

Culture media was removed from the wells and the adherent cells were fixed using ½ strength Karnovsky's fixative overnight at 4° C. Pre-embedding with heavy metals was performed according to the protocol developed by Deerinck et al. Conventional negative staining with 1% uranyl acetate (Electron Microscopy Sciences, 22400) was performed at 4° C. overnight. Thin sections with 60-90 nm thickness were prepared and examined using a JEOL JEM 2100 transmission electron microscope.

### Example 8

### VWF Multimer Gel Electrophoresis

On day 12 of cell differentiation, hPSC-EC were washed three times by EBM endothelial cell growth basal medium (Lonza). The cells were stimulated by 100 ng/mL Phorbol 12-myristate 13-acetate (PMA, Sigma Aldrich) at 37° C. and 5% CO₂ for 1 hour. Cell culture supernatants were collected and VWF multimer structures were analyzed by electrophoresis as previously described. Cell culture media containing VWF from HUVEC or recombinant VWF expressed from human embryonic kidney (HEK) 293 cells were used as positive controls.

VWF samples were loaded to a 1.7% SeaKem Gold agarose (Lonza) gel and ran at constant current 3 mA for ˜ 19 hours at room temperature. A western blot was performed using Tans-Blot SD semi-dry transfer apparatus (BioRad) for 1 hour and 15 minutes at constant 180 mA onto a PVDF 0.45 UM pore size membrane (BioRad). The membrane was then incubated with 0.5 μg/ml antibody for VWF (DAKO, A0082) in 5% milk-TBS (BioRad, 1× Tris-buffered saline) for 2 hours at room temperature followed by a secondary antibody goat anti-rabbit HRP (Invitrogen, 31460) at 0.1 μg/ml for 1 hour at room temperature in 5% milk-TBS. Afterwards, the membrane was incubated in chemiluminescent substrate for 5 minutes and VWF multimer profile was then imaged using ImageQuant 350 imager (GE healthcare life sciences).

### Example 9

Transfection of Plasmid Encoding VWF into VWF^{-/-} hPSC-EC

On d10 of cell differentiation, VWF^{-/-} hPSC-EC were passaged and seeded at 200,000 cells per well in a 24-well plate pre-coated with 0.1% gelatin (StemCell Technologies). On the next day, the cells were transfected with pcDNA3.1 plasmid encoding human VWF (SEQ ID NO: 14), using Lipofectamine Stem Transfection Reagent (Thermo Fisher Scientific). Mock transfection with pcDNA3.1 plasmid (empty vector) was used as a control. After 48 hours of transfection (d13 of cell differentiation), cells were fixed by 4% paraformaldehyde in 1×PBS for immunofluorescence imaging.

### Example 10

Regulation of VWF Expression Level in hPSC-EC

hPSC-EC were treated with blood plasma or compounds to test their effects on VWF expression level in cells. Either human whole blood plasma (Bloodworks Northwest, Seattle, USA) or VWF deficient plasma (Affinity Biologicals) was used to treat hPSC-EC. Blood plasma containing VWF was centrifuged at 2000 g for 5 minutes at room temperature and supernatant was used to treat the cells. The plasma was added at 5, 10, 20, 40, or 80% (v/v) of the total hPSC-EC culture medium on day 7 of the cell differentiation and maintained in the media thereafter. The cell culture medium with blood plasma was changed every two days until fixation or tube formation or VWF string formation experiments on day 10-14. Due to the high fluorescence background in 40% and 80% plasma treated cells, we only used less than 40% plasma to treat cells for immunofluorescence imaging.

Ten compounds were added separately to hPSC culture medium on day 6 of the cell differentiation and maintained in the media thereafter. The untreated cell culture medium was EGM with additional 20 ng/ml VEGF, 20 ng/ml FGF, and 1 μM Chir99021. Based on untreated cell culture medium, we tested the following additional concentrations of compounds: VEGF (30, 70, 120 ng/ml, Peprotech 100-20), FGF (30, 70, 120 ng/ml, PeproTech 100-18B), human transforming growth factor β1 (TGFβ1, 1, 3, 10 ng/ml, R&D Systems 240-B), lipopolysaccharide (LPS, 1, 3, 10 ng/ml, Santa Cruz sc-3535), human interferon gamma (IFN, 1, 3, 10 ng/ml, Peprotech 300-02), human noggin (1, 3, 10 ng/ml, Peprotech, 120-10C), Chir99021 (1, 3, 10 UM, Reprocell 04-0004-10), Smoothened agonist (SAG, 1, 3, 10 μM, Cayman Chemical 11914), BMP4 (1, 3, 10, 50 ng/ml, R&D Systems 314-BP), or DAPT (1, 3, 10 UM, Tocris 2634). Concentrations of these compounds that were likely to be safe and efficacious were used based on the literature and previous experience. To treat HUVEC with DAPT (Tocris 2634), 0/10/30 μM DAPT was added to the cell culture medium EGM on day 2 after cell passaging and maintained in the media thereafter. The cell culture medium with corresponding compound was changed every two days until cell fixation or cell lysis experiment on day 10-14 for hSPC-EC or day 7-9 for HUVEC.

### Example 11

### VWF String Formation Assay

The hPSC-EC from day 5 of cell differentiation were seeded in a flow chamber (ibidi GmbH, μ-slide VI 0.4, 17×3.8×0.4 mm) precoated with 2.5 mg/mL collagen (Corning, Rate Tail Collagen Type I). Cells were fed by EGM endothelial cell growth medium+20 ng/ml recombinant human VEGF₁₆₅+20 ng/mL recombinant human FGF-basic+1 μM Chir-99021 until day 12 of cell differentiation. The flow chamber was washed by 100 μl EBM endothelial cell growth basal medium (Lonza) twice. Cells were stimulated in EBM medium with 100 ng/mL PMA at stasis at 37° C. and 5% CO₂ for 20 minutes and then followed by flow (2.5 dyn/cm² wall shear stress) using a syringe pump for 5 minutes. After flow stopped, the flow chamber was blocked by 6% BSA (Sigma Aldrich) in EBM medium for 10 minutes. Cells were then stained by 1:100 diluted FITC-anti VWF antibody (Abcam; ab8822) in EBM medium with 6% BSA for 10 minutes. Cells were washed by EBM medium for three times and imaged under Olympus wide field fluorescence microscope. After imaging, cells were fixed, permeabilized, and blotted by antibodies for CD31 (BD Biosciences; 555444) or CD144 (BD Biosciences; 555661) for immunofluorescence imaging of endothelial cells.

### Example 12

### Statistical Analysis

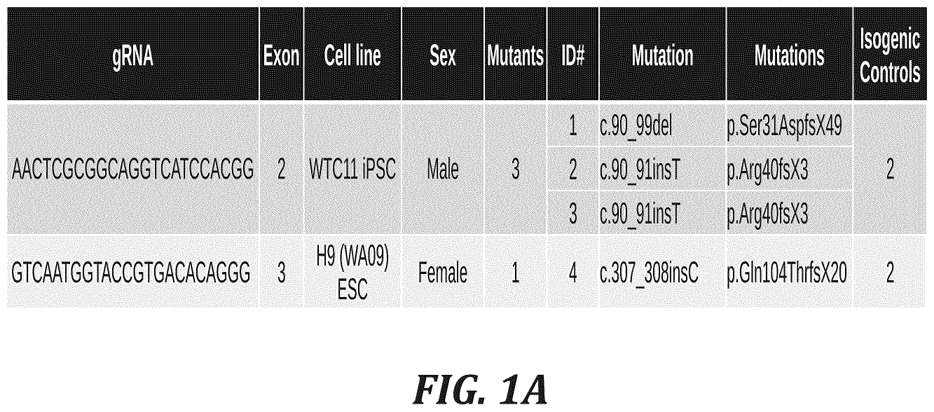

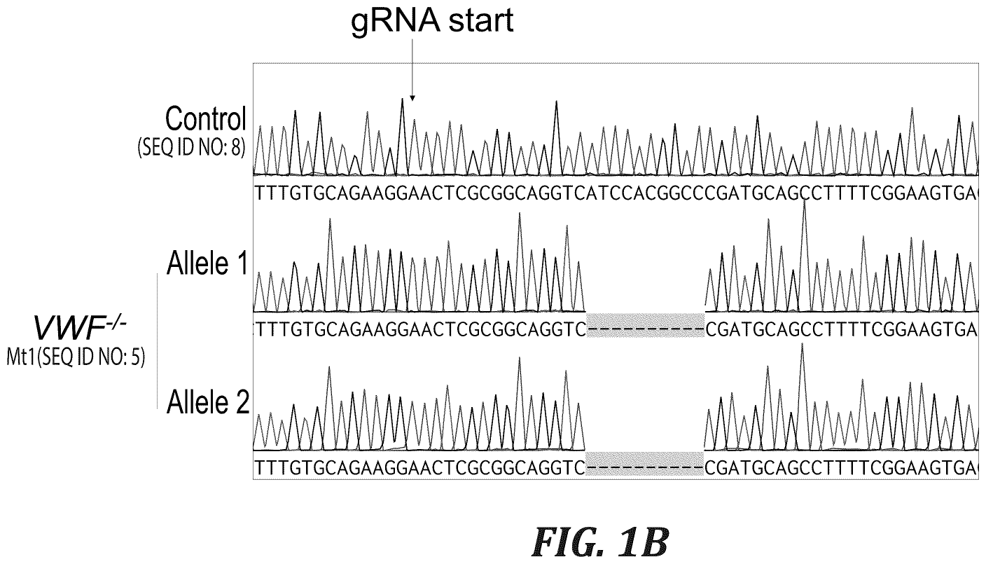

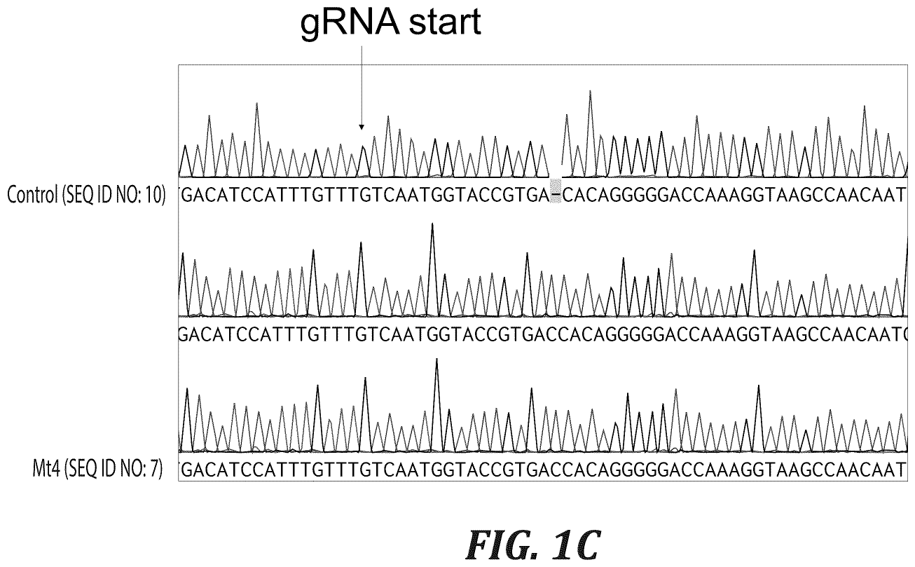

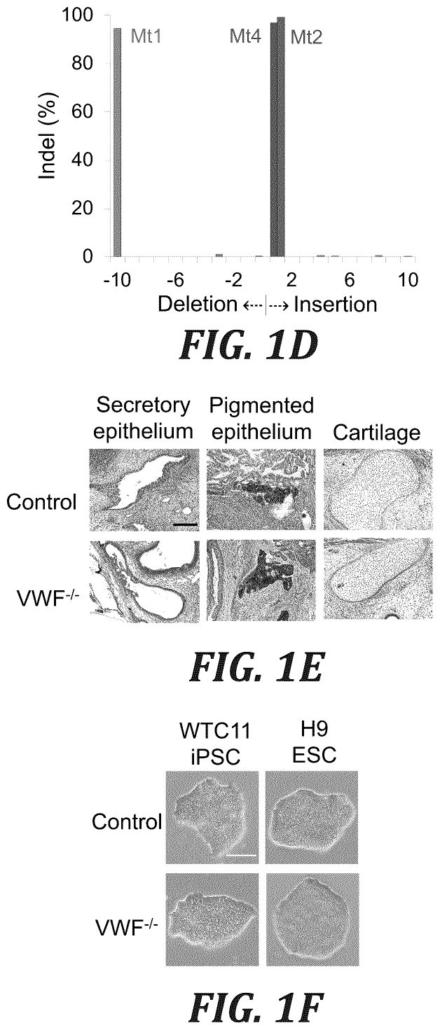

Experiments were performed using a cohort of hPSC, including one parental WTC11 iPSC line, four isogenic control lines for WTC11 iPSC and H9 ESC that were subjected to CRISPR mutagenesis but were found to be unmodified at the targeted locus, and four VWF^{-/-} lines (FIG. 1A). Quantification was performed on at least three independent experiments. Error bars are mean±standard error (s.e.m). Statistical analyses were performed using GraphPad Prism Software. To test significance, p values were calculated using two-tailed, unpaired t-test for samples with unequal variances. A p value <0.05 (*p<0.05, **p<0.01, ***p<0.001, ****p<0.0001) was considered significant for all tests. For samples with multiple conditions (e.g. dose escalation studies), ANOVA tests were performed, unless otherwise noted.

### Example 13

Generation of VWF-Deficient Endothelial Cells from Gene Edited hPSCs

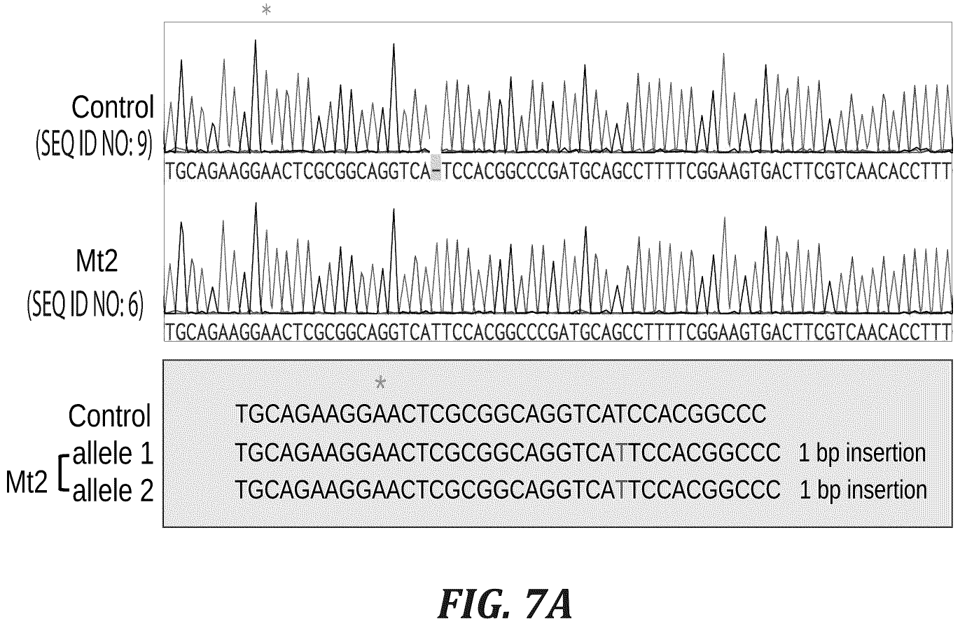

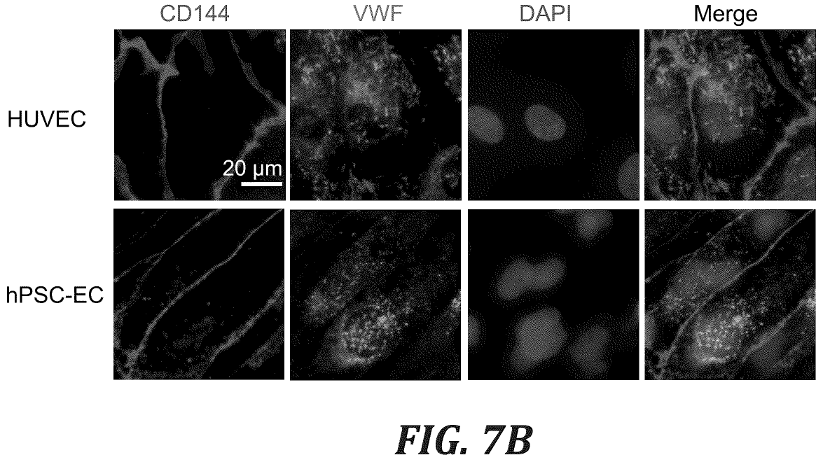

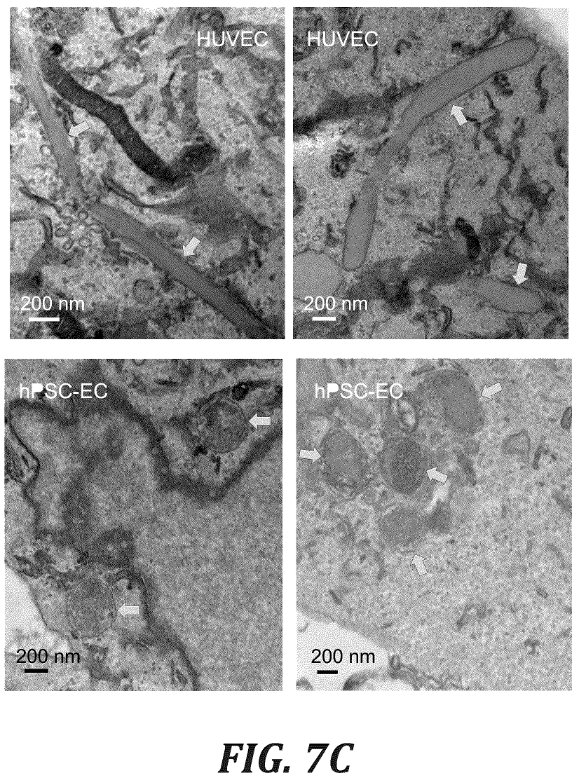

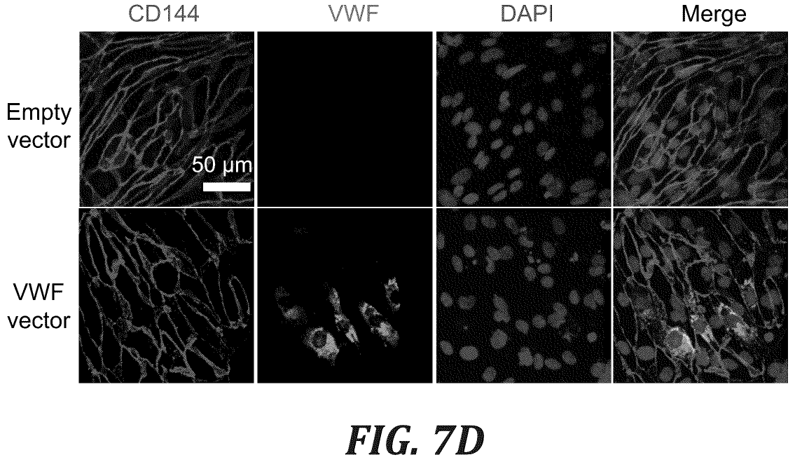

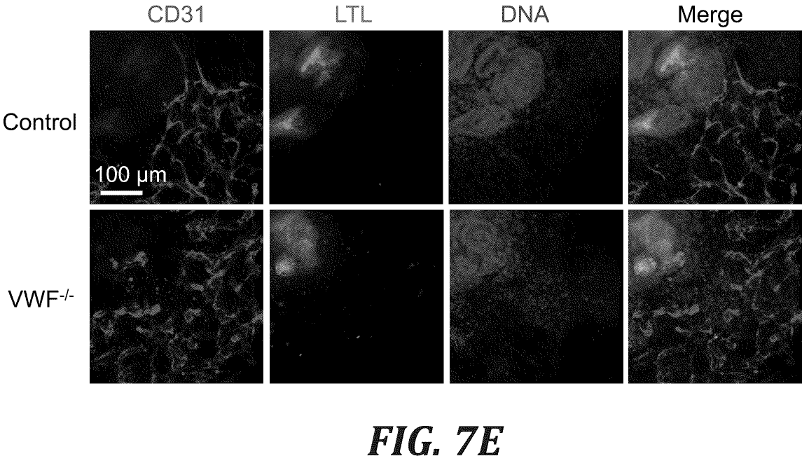

To develop a human cellular model of VWF deficiency, CRISPR-Cas9 genome editing system was used to disrupt the VWF gene in hPSC. This process produced a cohort consisting of four VWF-deficient (VWF^{-/-}) cell lines and four non-mutant isogenic controls. Characteristics of this cell line cohort are summarized in FIG. 1A. To control potential off-target effects of the editing, two distinct guide RNAs (gRNA) (SEQ ID NO: 2 and SEQ ID NO: 3) were used separately to generate cell lines with insertion or deletion mutations in either exon 2 or exon 3. Each gRNA was designed to have minimal off-target effects on the genome. Two distinct parental cell lines, one male and one female, were utilized to enable comparison across different genetic backgrounds. Frame-shift mutations at the gRNA target site were detected by DNA sequencing of PCR amplicons and confirmed by cloning and sequencing of the individual alleles as well as computational analysis (FIGS. 1B-1D and FIG. 7A). VWF^{-/-} cell lines maintained a cellular morphology indistinguishable from isogenic controls over many passages in vitro and differentiated into teratomas containing representative tissues of the three embryonic germ layers when implanted into immunodeficient mice (FIGS. 1E-IF).

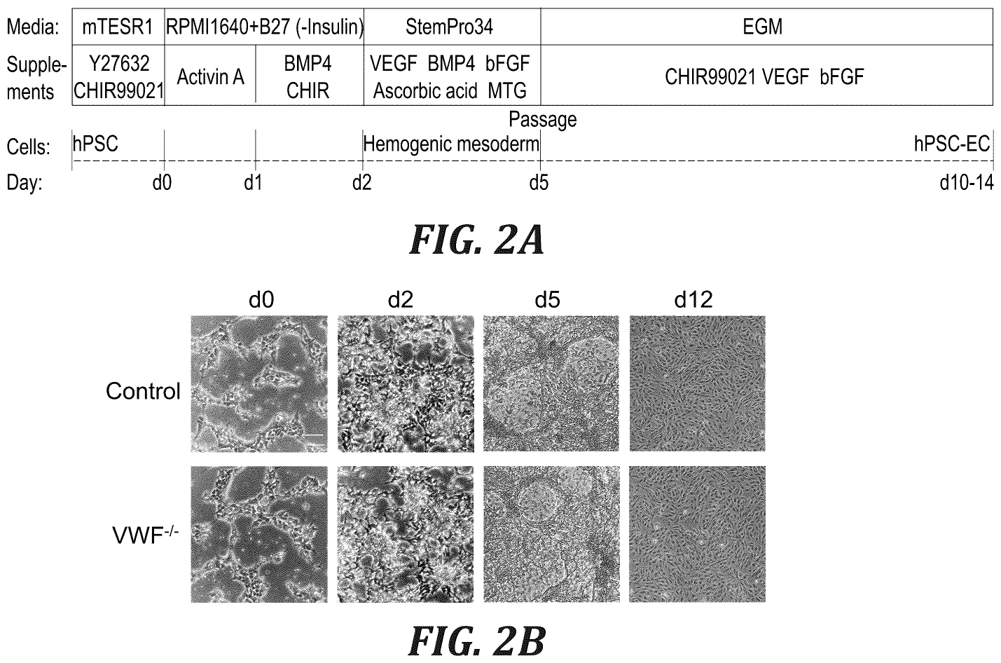

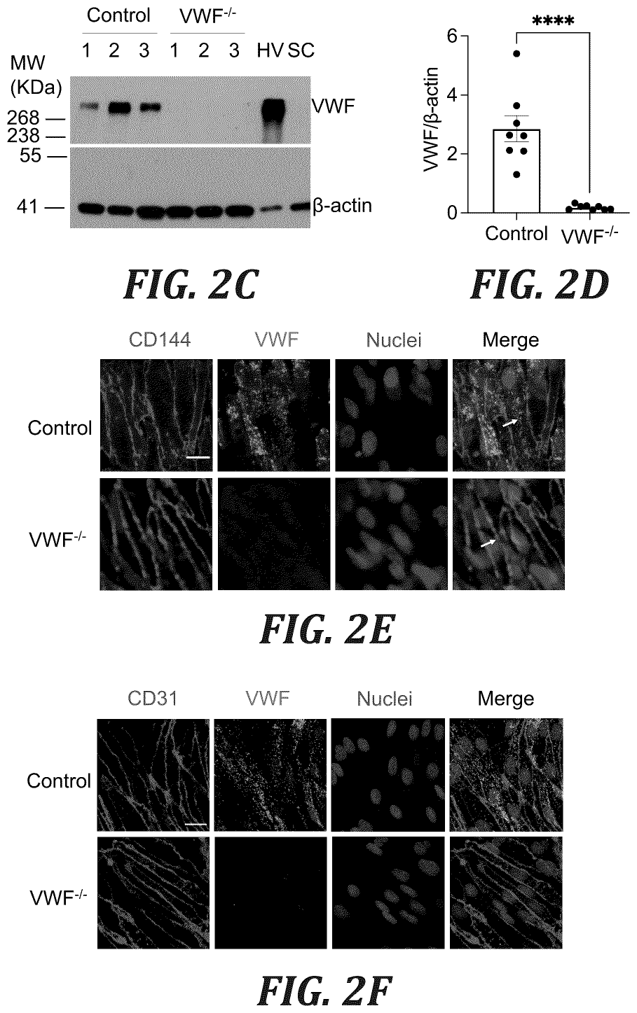

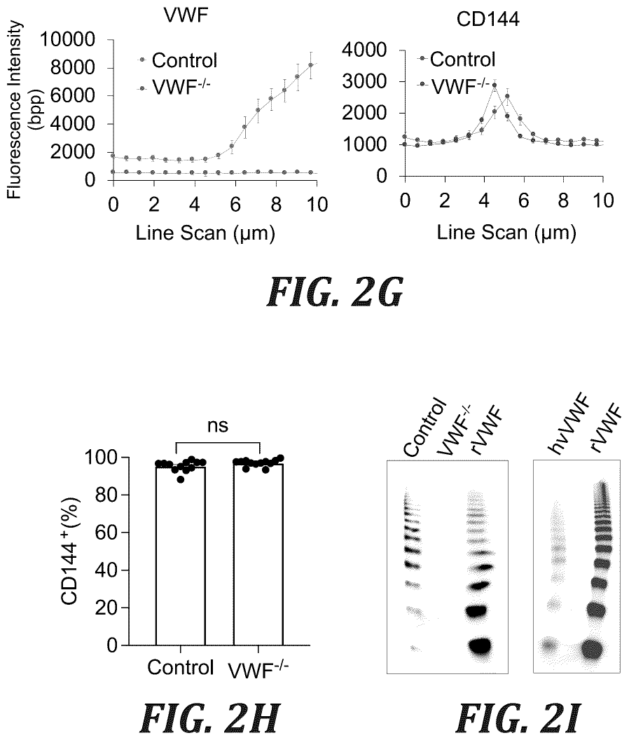

To study VWF gene expression, both VWF^{-/-} and control hPSC were differentiated into endothelial cells (hPSC-EC) using a previously established differentiation protocol (FIG. 2A). This produced monolayers of elongated, polygon-shaped cells with similar morphologies in both VWF^{-/-} and control cultures (FIG. 2B). VWF was readily detected by immunoblot in control hPSC-EC, but was undetectable in VWF/hPSC, confirming that the indel mutations had successfully disrupted the coding sequence of the gene (FIGS. 2C-2D). The expression levels of VWF in control hPSC-EC were noted to be generally lower than in human umbilical vein endothelial cells (HUVEC, Lonza) (FIG. 2C). In control hPSC-EC, VWF was detected by immunofluorescence in punctate foci surrounding the nucleus, reminiscent of WPBs but less rod-shaped than corresponding structures in HUVEC (FIGS. 2E-2F and FIG. 7B). Round-shaped WPB-like structures in hPSC-EC were also observed by electronic microscopy imaging (FIG. 7C). In contrast, specific immunofluorescence for VWF was not detected in VWF^{-/-} hPSC-EC (FIGS. 2E-2F).

Other EC markers, including CD144 and CD31, were expressed in similar quantities and localization patterns in both VWF^{-/-} and control hPSC-EC (FIGS. 2E-2F). Quantitative line scan and fluorescence intensity analysis of hPSC-EC populations further demonstrated a specific deficiency in levels of VWF, but not CD144 (FIGS. 2G-2H). Approximately 95% of the cells in these cultures expressed CD144 (FIG. 2H). VWF was secreted into the supernatants of control hPSC-EC with a multimerization pattern similar to VWF expressed in HEK293 cells and HUVEC, whereas multimers were absent in supernatants of VWF^{-/-} mutants (FIG. 2I). Thus hPSC-EC exhibited the ability to express and store VWF in WPBs and secrete VWF as multimers. When transfected with a rescue construct encoding human VWF, VWF^{-/-} hPSC-EC efficiently expressed exogenous VWF, as detected by immunofluorescence (FIG. 7D). To generate EC using a different method, a kidney organoid differentiation protocol that enriches for EC was followed. The results showed that VWF loss similarly had no effect on EC differentiation in kidney organoid cultures derived from VWF/hPSC (FIG. 7E).

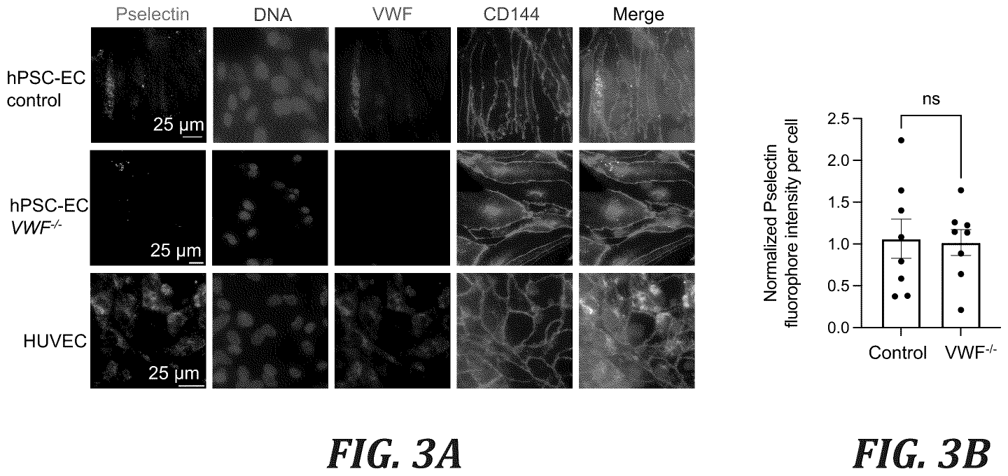

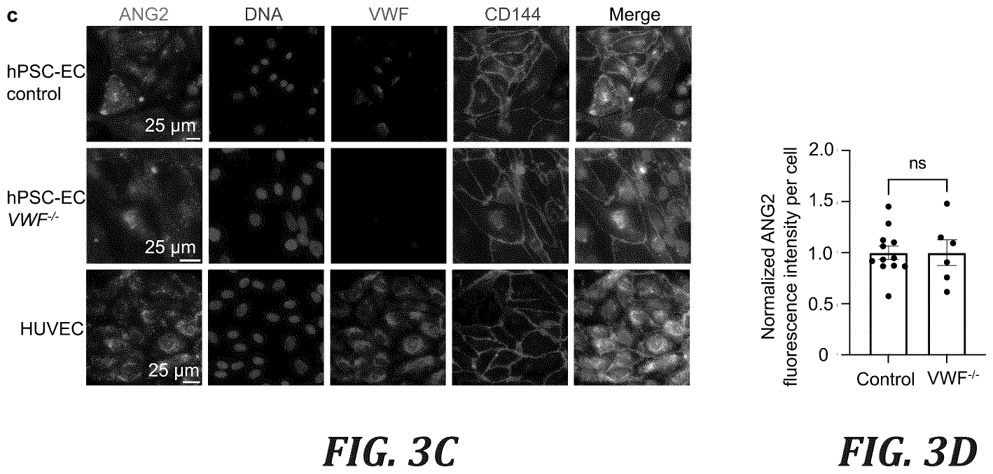

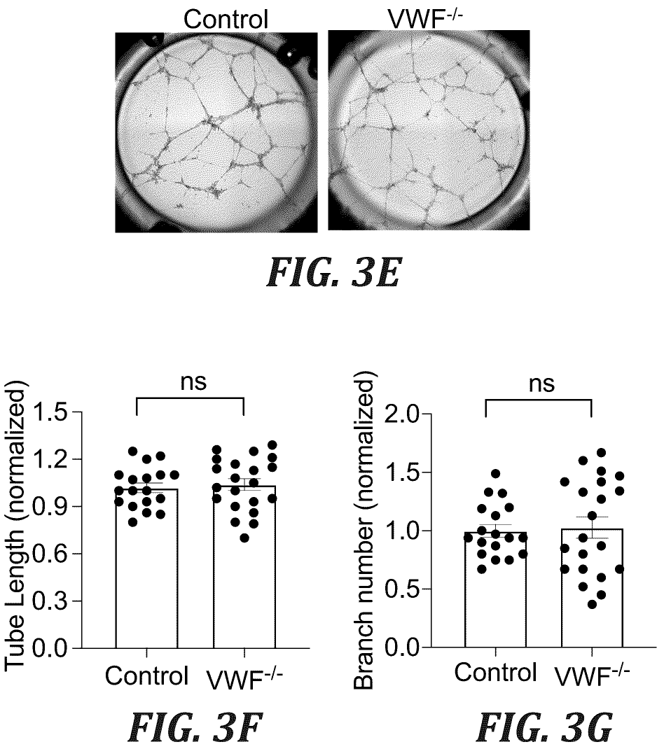

Besides VWF, P selectin and angiopoietin 2 (ANG2) are known to be expressed in WPB. P selectin and ANG2 were expressed in both VWF^{-/-} and isogenic control hPSC-EC (FIG. 3). P selectin was colocalized with VWF inside WPBs in control hPSC-EC, but with much lower expressing amount (FIGS. 3A-3B). ANG2 in hPSC-EC was not exclusively located in WPBs as those in HUVEC (FIG. 3C). No significant differences of ANG2 and P selectin expression levels were detected from immunofluorescence imaging in hPSC-EC VWF^{-/-} cell lines, compared to isogenic controls (FIG. 3B, FIG. 3D). Collectively, these findings demonstrated that VWF was dispensable for hPSC differentiation into EC and its expression of P selectin and ANG2. ANG2 has been shown to promote capillary-like structures in EC via VEGF and other pathways. Both control and VWF/hPSC-EC could form capillary-like structures in Matrigel without significant difference in tube length or branch number (FIGS. 3E-3G).

### Example 14

Blood Plasma Enhances hPSC-EC Maturation

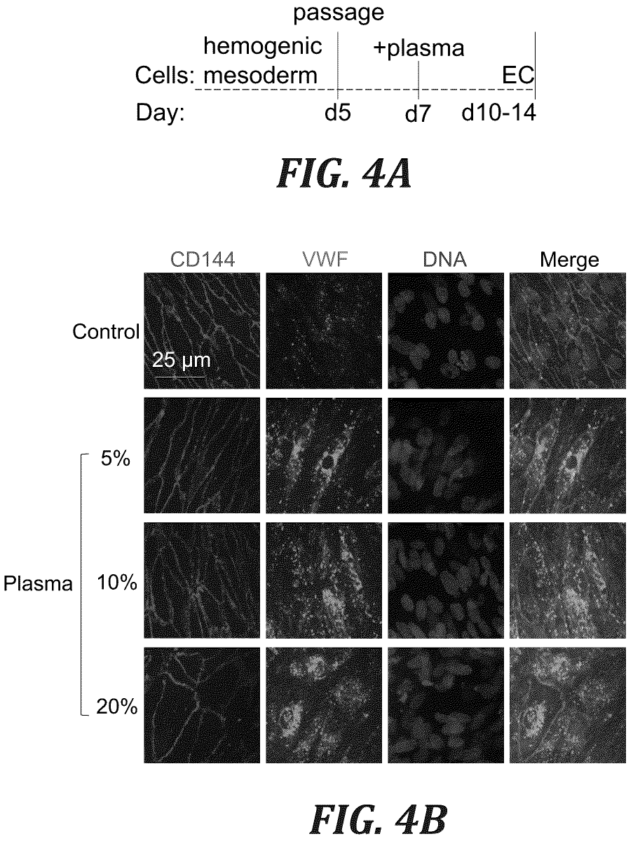

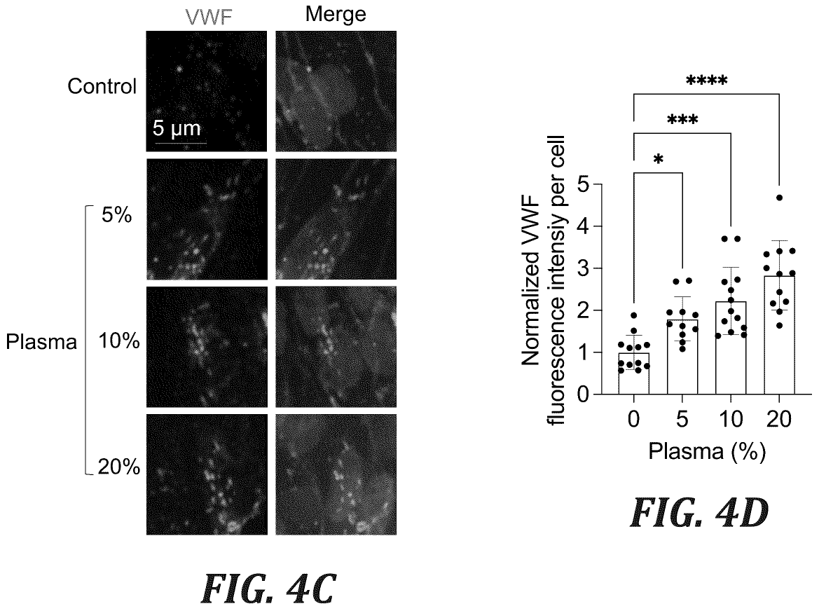

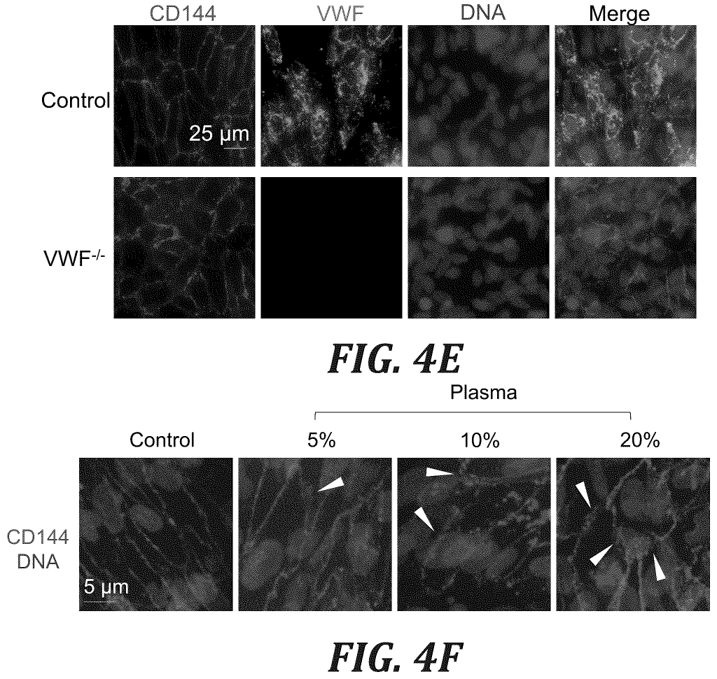

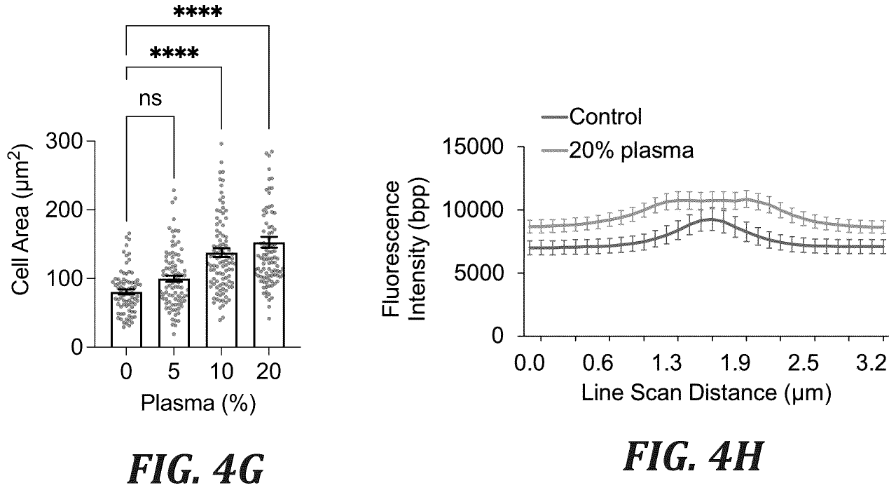

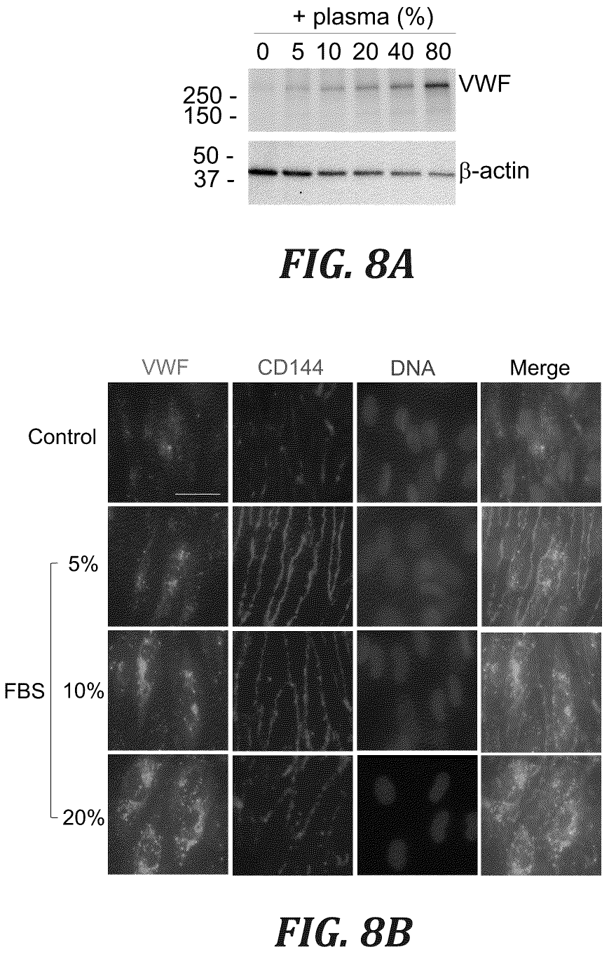

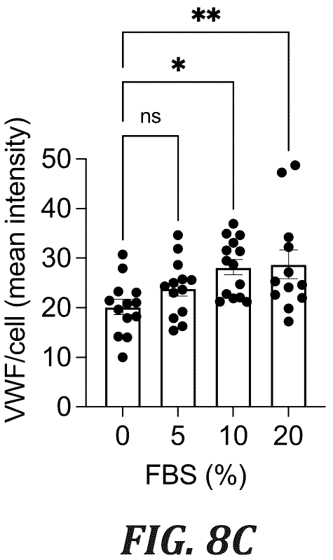

Although hPSC-EC expressed VWF, we found these cells were difficult to form VWF strings as HUVEC did. As shown in FIG. 2C, VWF levels in hPSC-EC were lower than in HUVEC, which might affect the amount of secreted VWF and be responsible for string formation trouble. To develop a robust cell model to study VWF, the inventors therefore sought to identify conditions capable of augmenting VWF expression in hPSC-EC. As EC are naturally exposed to blood during embryonic development and throughout adult life, the inventors evaluated whether blood might serve as a maturation cue. Human plasma was added to cultures on day 7 of differentiation, a time point that immediately preceded terminal differentiation, and maintained thereafter in these cultures (FIG. 4A). Strikingly, WPBs containing VWF were noticeably brighter and more abundant with increasing concentrations of plasma (FIG. 4B and FIGS. 8A-8B). These treatments also produced more rod-shaped WPBs, compared to the punctate structures typical of differentiations without plasma (FIG. 4C). Increasing concentrations showed dose-dependent effects that were significantly different from the control, with 20% plasma increasing VWF intensity by approximately three-fold (FIG. 4D). Similarly, addition of fetal bovine serum (FBS, Lonza) up to 20% of total volume also resulted in significant and dose-dependent increases in VWF fluorescence intensity levels, although the effect was more modest compared to the previous experiments using plasma (FIG. 8B-8C). These experiments revealed that soluble factors within the blood can substantially augment VWF expression in differentiating hPSC-EC.

Blood plasma contains VWF, raising the possibility that the increase in VWF staining was due to endocytosis. To test this, the same experiments were performed in the VWF^{-/-} hPSC-EC, but no detectable VWF was observed in these cells (FIG. 4E). Thus, the enhanced VWF fluorescence intensity in control EC reflected the ability of plasma to induce intracellular VWF expression, rather than endocytosis, which was also confirmed by VWF deficient plasma treated cells.

In addition to its effects on VWF, plasma had pronounced effects on hPSC-EC suggestive of improved maturation. hPSC-EC treated with plasma were significantly larger than untreated hPSC-EC (FIGS. 4F-4G). CD144⁺ junctional complexes in treated cells were noticeably thicker than in untreated cells and exhibited lattice-shaped adherens junctions characteristic of mature EC in culture (FIG. 4F, FIG. 4H). These features suggested that plasma generally improves the maturation state of hPSC-EC.

### Example 15

A Small Molecule DAPT Enhances VWF Expression in Both hPSC-EC and HUVEC

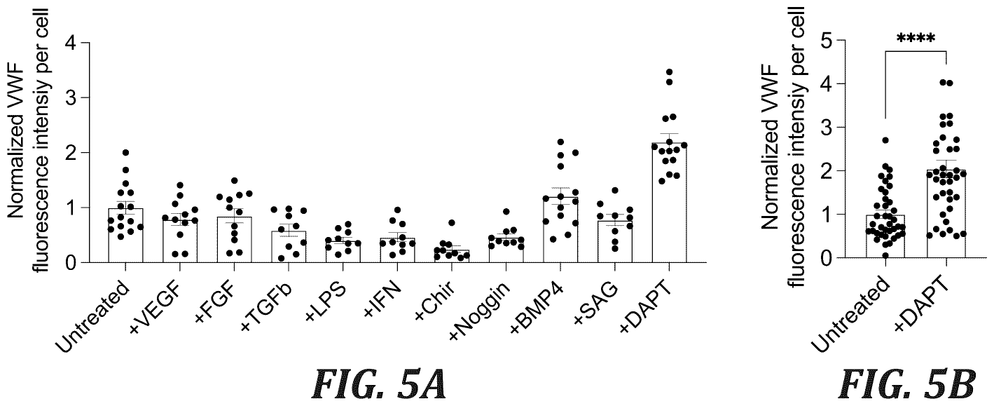

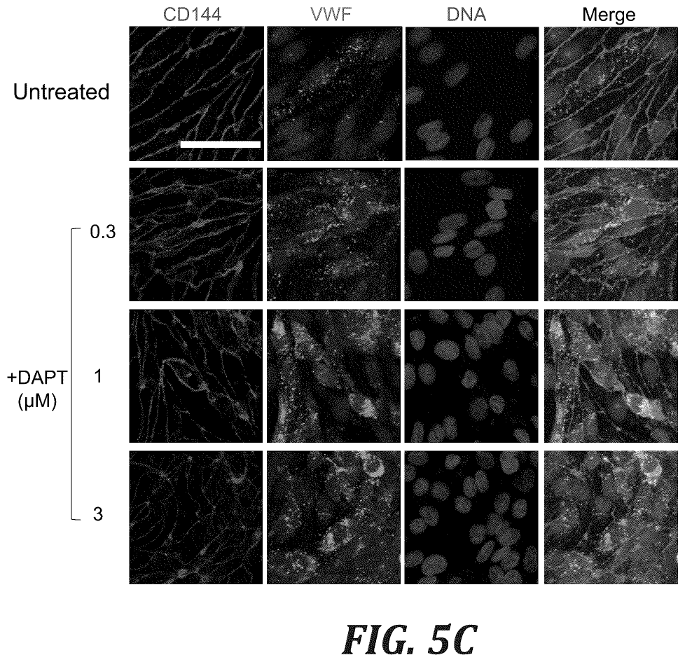

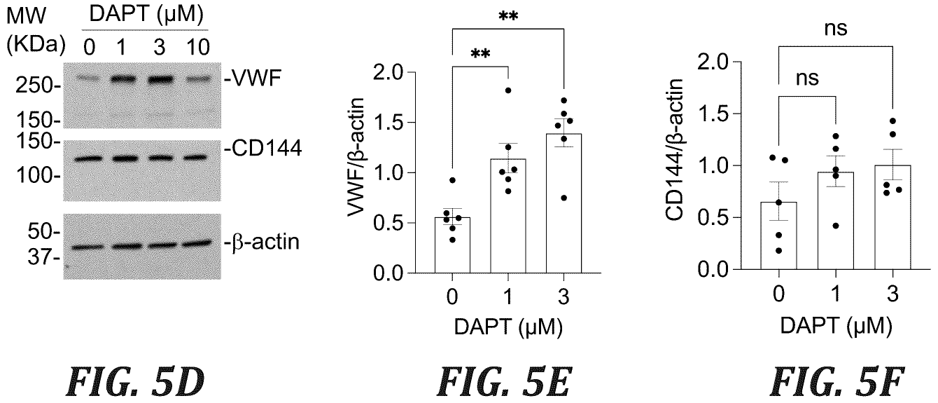

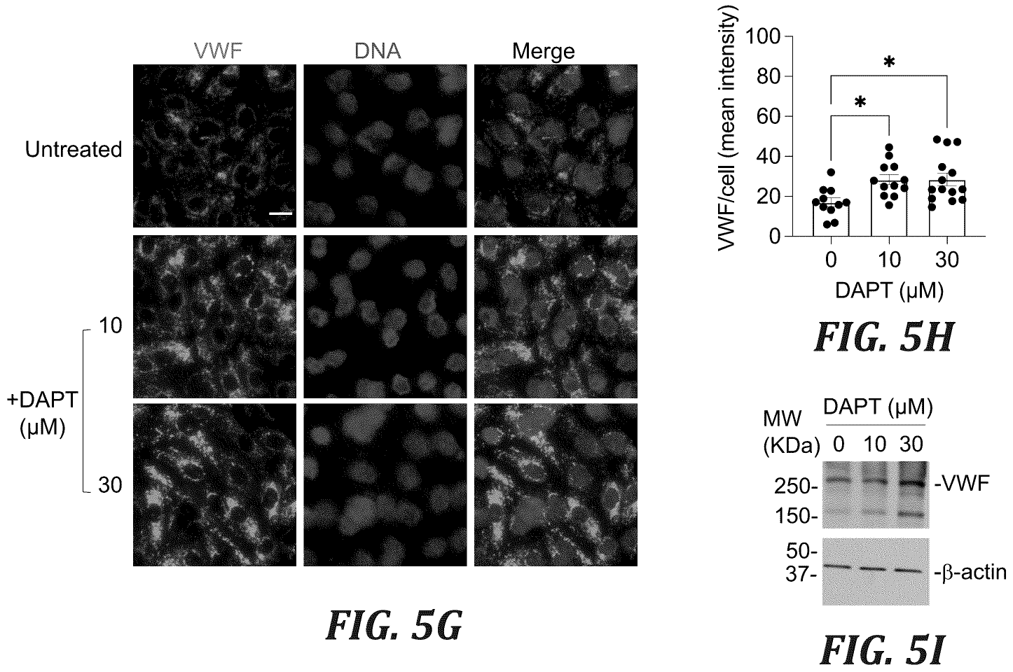

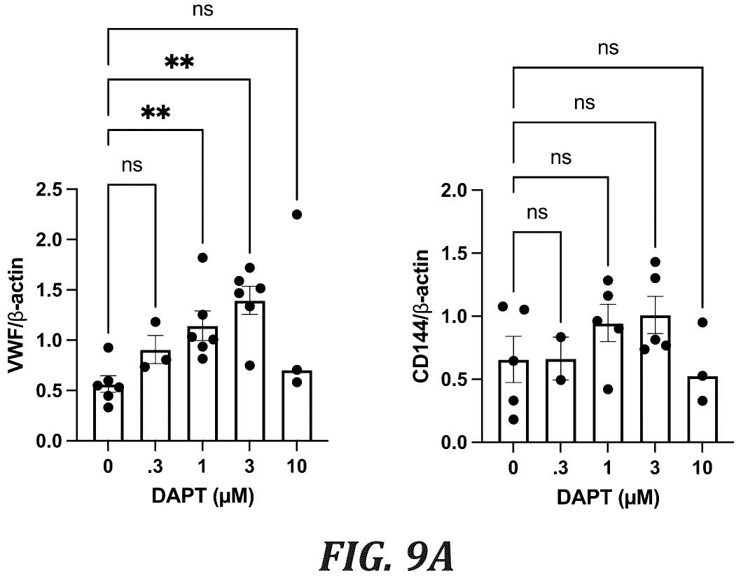

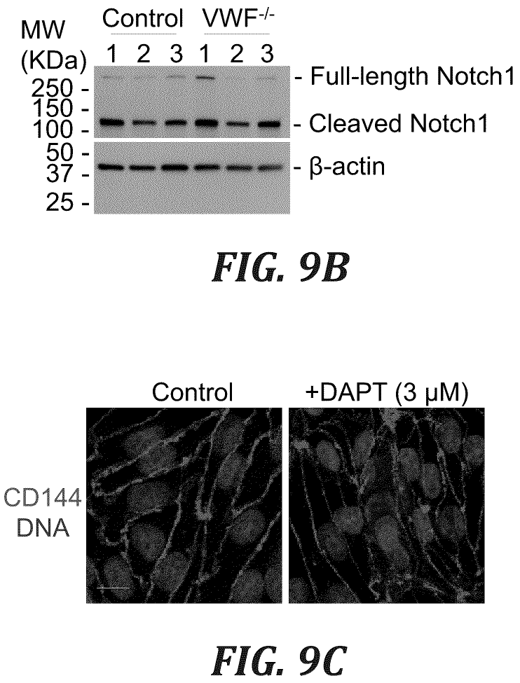

For stem cell differentiation, defined chemical factors are preferable to complex, undefined additives such as FBS or plasma. To identify mechanistic pathways and determine whether defined factors might also enhance VWF expression level, we screened ten compounds relevant to hPSC-EC differentiation for their ability to enhance VWF expression. These compounds were added separately on day 6 of our differentiation protocol and maintained in the media thereafter. One of these, the gamma secretase inhibitor DAPT, substantially increased levels of VWF, as detected by immunofluorescence (FIG. 5A). Repeated trials revealed that DAPT increased VWF approximately two-fold in this assay, when added at a final concentration of 1 μM (FIGS. 5B-5C). Immunoblot analysis confirmed the increase in VWF protein in DAPT-treated cells, peaking at ˜3 μM final concentration, whereas higher concentrations were less beneficial, likely due to off-target effects of the drug (FIGS. 5D-5F and FIG. 9A). Notch1, a potential downstream target of DAPT, was expressed in hPSC-EC by immunoblot in both full-length and cleaved forms (FIG. 9B). DAPT is known to inhibit gamma secretase, and therefore block Notch signaling, which can affect gene expression patterns in EC. Inhibition of Notch thus might be responsible to the increase of VWF expression level.

Next it was determined whether the effect of DAPT on VWF was caused by modulating stem cell differentiation, or whether it could also happen in terminally differentiated (primary culture) EC. When HUVEC were treated with DAPT, immunofluorescence and immunoblot analysis both showed enhanced VWF level with increased DAPT concentrations (FIGS. 5G-5I). Compared to untreated controls, VWF increased ˜1.7 times in the presence of 10 μM DAPT. These results show that DAPT can upregulate VWF expression in both primary EC and hPSC-EC. DAPT did not induce the changes in hPSC-EC size or junctional morphology observed previously with plasma treatment (FIG. 9C).

### Example 16

### Increased VWF Level Enables VWF String Formation

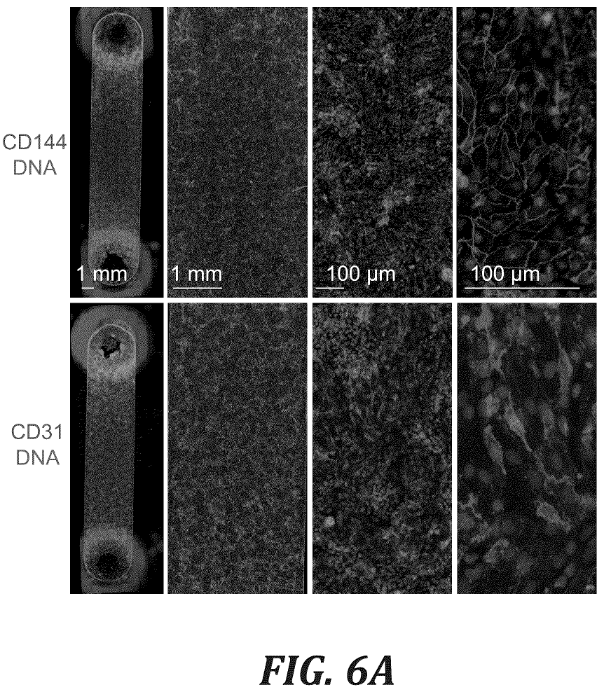

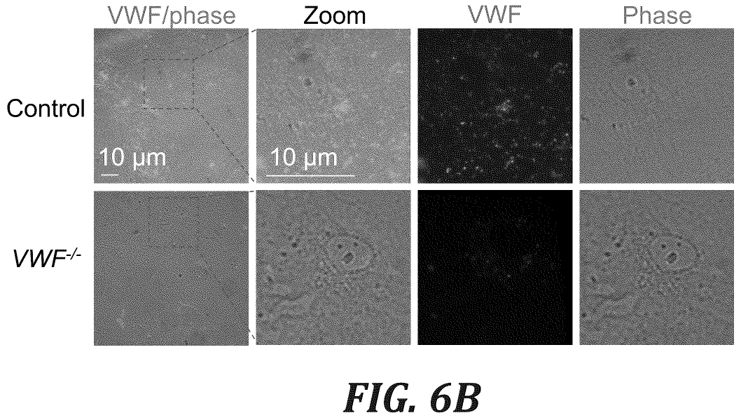

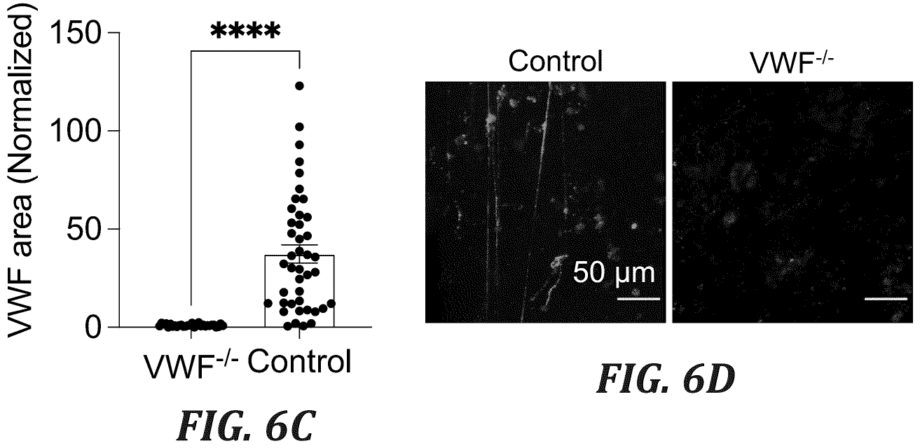

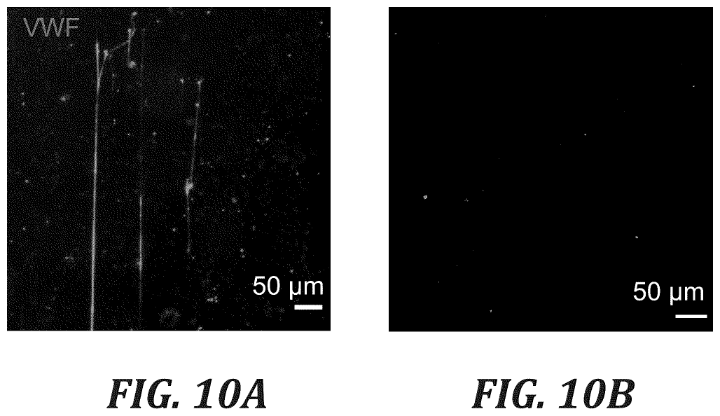

VWF forms strings on HUVEC surface upon stimulation by agonists such as phorbol 12-myristate 13-acetate (PMA) (FIG. 10A). However, it was difficult to detect VWF at the cell surface after hPSC-EC stimulation with PMA using standard differentiation protocol (FIG. 10B). Possible causes for this included insufficient VWF expression, or a deficiency in WPBs associated with their unusual round shape in hPSC-EC, which differs from the rod shape typical of WPBs in primary EC. The effect of enhancing VWF level on VWF string formation was then evaluated. hPSC-EC were transferred into a microfluidic channel on day 5 of differentiation and treated with 3 μM DAPT from day 6 onwards. On day 12-14, hPSC-EC formed confluent monolayers expressing CD144 and CD31 (FIG. 6A). To stimulate VWF secretion, hPSC-EC in these channels were first treated with 100 ng/mL PMA for 20 minutes under stasis, followed by flow at a shear stress of 2.5 dyn/cm² for 5 minutes. VWF attached to the EC surface was detected live by FITC-labeled anti-VWF antibody, which was introduced under flow. This revealed numerous foci of VWF at the cell surface of control hPSC-EC, but not in VWF/hPSC-EC (FIG. 6B-6C). Some of the secreted VWF formed strings attached to the EC surface (FIG. 6D). VWF^{-/-} hPSC-EC were essential controls in these experiments, enabling us to distinguish the specific VWF signal from background. These results indicated that enhanced VWF expression can improve VWF secretion and enable VWF string formation at the cell surface.

The complete disclosure of all patents, patent applications, and publications, and electronically available material cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

The description of embodiments of the disclosure is not intended to be exhaustive or to limit the disclosure to the precise form disclosed. While illustrative embodiments have been illustrated and described, it will be appreciated that various changes can be made therein without departing from the spirit and scope of the disclosure.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural and singular number, respectively. Additionally, the words “herein,” “above,” and “below” and words of similar import, when used in this application, shall refer to this application as a whole and not to any particular portions of the application.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

The following numbered paragraphs (paras), describing aspects of the present disclosure, are part of the description.
1. A method of generating a cellular model of VWF disease, the method comprising (i) providing at least one pluripotent stem cell; (ii) contacting the at least one pluripotent stem cell with an agent effective in suppressing or deleting at least one gene sequence or a portion thereof encoding a protein or a subunit thereof, wherein the protein or the subunit thereof is associated with VWF linked protein secretion; and (iii) differentiating said pluripotent stem cell into an endothelial cell.
2. The method of para 1, wherein the at least one pluripotent stem cell is a human pluripotent stem cell (hPSC).
3. The method of para 2, wherein the hPSC is a human embryonic stem cell (ES).
4. The method of para 1 wherein the pluripotent stem cell is an induced pluripotent stem cell.
5. The method of para 1, wherein the agent is effective in suppressing or deleting at least one gene sequence or a portion thereof encoding at least one protein, or a subunit thereof, associated with VWF-linked protein secretion.
6. The method of para 5, wherein the at least one gene sequence comprises the VWF gene, and wherein the agent is effective in disrupting the VWF gene to produce a pluripotent stem cell-derived endothelial cell with knock out mutation of VWF.
7. The method of para 6, wherein the agent effective in disrupting the VWF gene comprises CRISPR/Cas9 genome editing system comprising nucleotide sequences encoding CRISPR-Cas guide RNAs, wherein the guide RNAs hybridize with a target sequence in exon 1 or in exon 2 of the VWF gene.
8. The method of para 7, wherein the CRISPR-Cas guide RNAs are selected from SEQ ID NO:2 or SEQ ID NO:3.
9. The method of para 1, further comprising contacting the endothelial cell with an agent effective in enhancing the expression levels of VWF; restoring the expression of VWF; restoring VWF-linked protein secretion, and/or restoring VWF string formation.
10. An endothelial cell produced by the method of para 1, wherein the endothelial cell is deficient in VWF (von Willebrand factor gene).
11. An in vitro-generated human endothelial cell differentiated from a human pluripotent stem cell (hPSC-EC).
12. The human endothelial cell of para 11, wherein the hPSC-EC is deficient in at least one gene sequence associated with VWF-linked protein secretion.
13. The human endothelial cell of para 12, wherein the hPSC-EC lacks the VWF (von Willebrand factor gene).
14. The human endothelial cell of para 13, wherein the hPSC-EC lacking VWF (i) has a morphology similar to a human pluripotent stem cell differentiated into endothelial cell (hPSC-EC) comprising VWF; and/or (ii) forms capillary-like structures with similar tube length and branch numbers as formed by hPSC-EC comprising VWF; and/or (iii) expresses CD144, CD31, P-selectin, and ANG2 in similar quantities and in similar localization patterns as expressed by hPSC-EC comprising VWF.
15. The human endothelial cell of para 13, wherein the hPSC-EC lacking VWF does not secrete VWF multimers.
16. The human endothelial cell of para 11, wherein the hPSC-EC (i) expresses CD144, CD31, P-selectin, and ANG2; (ii) expresses VWF stored in WPBs; (iii) forms capillary-like structures; and (iv) secretes VWF multimers.
17. The human endothelial cell of para 11, para 12, or para 13, wherein the endothelial cell is capable of forming a cell line, a teratoma, a tissue, or an organoid.
18. A cell line generated from the hPSC-EC of para 17.
19. An organoid generated from the hPSC-EC of para 17.
20. The cell line of para 18, wherein the cell line models a feature of blood disorder selected from: thrombosis, deep vein thrombosis, blood clots, stroke, heart disease and von Willebrand disease.
21. The organoid of para 19, wherein the organoid models a feature of blood disorder selected from: thrombosis, deep vein thrombosis, blood clots, stroke, heart disease and von Willebrand disease.
22. A microfluidic culture device comprising cells obtained from the cell line of para 17.
23. A microfluidic device comprising the organoid of para 19.
24. A high throughput method for identifying a therapeutic agent for treating a bleeding disorder, the method comprising: contacting a plurality of candidate agents with cells obtained from the cell line of para 18, wherein an agent effective in: enhancing the expression levels of VWF; restoring the expression of VWF; restoring VWF-linked protein secretion, and/or restoring VWF string formation, is identified as the therapeutic agent effective for treating bleeding disorders.
25. The method of para 24, wherein the therapeutic agent is selected from: a genetic agent, a component of the cell culture, or a small molecule agonist of VWF expression.
26. The method of para 25, wherein the genetic agent is selected from a vector comprising a polynucleotide encoding VWF protein or a derivative thereof, or a polynucleotide encoding a protein linked with VWF secretion or a derivative thereof.
27. The method of para 25, wherein the component of the cell culture comprises blood plasma.
28. The method of para 25, wherein the small molecule agonist of VWF expression comprises DAPT.
29. The method of para 24, wherein the microfluidic device comprises an organ-on-chip configured to represent VWF disease.
30 The method of para 24 further comprising assessing toxicity of the therapeutic agent.
31. The method of para 24, wherein the bleeding disorder is selected from: thrombosis, deep vein thrombosis, blood clots, stroke, heart disease and von Willebrand disease.
32. A kit comprising the cell line of para 18.
33. A kit comprising the organoid of para 19.
34. A composition comprising at least one isolated human pluripotent stem cell differentiated into an endothelial cell (hPSC-EC) of para 13.
35. A method of generating one or more human pluripotent stem cell lines comprising the steps of: identifying at least one protein linked to VWF protein secretion in a biological sample; contacting a plurality of hPSCs with an agent effective in suppressing or deleting at least one gene sequence encoding the identified protein or a subunit thereof; disrupting a VWF-linked protein secretion pathway in the plurality of hPSCs; identifying modified hPSCs with disrupted VWF-linked protein secretion pathway; expanding the modified hPSCs to obtain a plurality of modified hPSCs as a clonal population; and deriving a cell lineage or tissue from the modified hPSCs clonal population to obtain endothelial cells.
36. The method of para 35 further comprising an additional step of stimulating endothelial cell specific organelles.
37. The method of para 36, wherein the specific organelles are Weibel-Palade bodies (WPBs).
38 The method of para 35, wherein the identified protein is VWF, and wherein VWF is identified using antibodies specific to a multi-lineage phenotype.
39. The method of para 35, wherein the disrupting step changes an amount of VWF linked protein secretion.
40. The method of para 35, wherein the gene sequence encodes VWF protein, and wherein the suppressing or deleting step is performed by gene editing.
41. The method of para 35, wherein the method produces VWF deficient mutants.
42. The method of para 35, wherein the one or more human pluripotent stem cell line models a feature of blood disorder selected from: thrombosis, deep vein thrombosis, blood clots, stroke, heart disease and von Willebrand disease.
43. The method of para 35, further comprising the step of generating non-mutant isogenic human pluripotent stem cell derived endothelial cell (hPSC-EC) control cell lines with intact VWF-linked protein secretion.
44 The method of para 35, wherein the cell line is used in a biochemical assay or a pharmaceutical kit.
45. An organoid comprising one or more cell lines of para 35.
46. A microfluidic culture device comprising cells obtained from cell lines of para 35.
47. The microfluidic culture device of para 46, wherein the device comprises an organ-on-a-chip culture device.
48. A method of treating a bleeding disorder in a subject in need thereof, the method comprising: administering to the subject an effective amount of a therapeutic agent identified by the method of para 24, wherein the therapeutic agent is effective in: (i) enhancing the expression levels of VWF; (ii) restoring the expression of VWF; (iii) restoring VWF-linked protein secretion, and/or (iv) restoring VWF string formation.
49 The method of para 48, wherein the bleeding disorder is selected from: thrombosis, deep vein thrombosis, blood clots, stroke, heart disease and von Willebrand disease.

1. A method of generating a cellular model of VWF disease, the method comprising: (i) providing at least one pluripotent stem cell; (ii) contacting the at least one pluripotent stem cell with an agent effective in suppressing or deleting at least one gene sequence or a portion thereof encoding a protein or a subunit thereof, wherein the protein or the subunit thereof is associated with VWF linked protein secretion; and (iii) differentiating said pluripotent stem cell into an endothelial cell.

2. The method of claim 1, wherein the at least one pluripotent stem cell is a human pluripotent stem cell (hPSC).

3. The method of claim 2, wherein the hPSC is a human embryonic stem cell (ES).

4. The method of claim 1 wherein the pluripotent stem cell is an induced pluripotent stem cell.

5. The method of claim 1, wherein the agent is effective in suppressing or deleting at least one gene sequence or a portion thereof encoding at least one protein, a subunit thereof, or a derivative thereof, associated with VWF-linked protein secretion.

6. The method of claim 5, wherein the at least one gene sequence comprises the VWF gene or a portion thereof, and wherein the agent is effective in disrupting the VWF gene to produce a pluripotent stem cell-derived endothelial cell with knock out mutation of VWF.

7. The method of claim 6, wherein the agent effective in disrupting the VWF gene or a portion thereof comprises: CRISPR/Cas9 genome editing system comprising nucleotide sequences encoding CRISPR-Cas guide RNAs, wherein the guide RNAs hybridize with a target sequence in exon 1 or in exon 2 of the VWF gene.

8. The method of claim 7, wherein the CRISPR-Cas guide RNAs are selected from SEQ ID NO:2 or SEQ ID NO:3.

9. An endothelial cell produced by the method of claim 1, wherein the endothelial cell is deficient in VWF (von Willebrand factor gene).

10. An in vitro-generated human endothelial cell differentiated from a human pluripotent stem cell (hPSC-EC).

11. The human endothelial cell of claim 10, wherein the hPSC-EC is deficient in at least one gene sequence associated with VWF-linked protein secretion.

12. The human endothelial cell of claim 11, wherein the hPSC-EC lacks the VWF (von Willebrand factor gene).

13. The human endothelial cell of claim 12, wherein the hPSC-EC lacking VWF:
(i) has a morphology similar to a human pluripotent stem cell differentiated into endothelial cell (hPSC-EC) comprising VWF; and/or
(ii) forms capillary-like structures with similar tube length and branch numbers as formed by hPSC-EC comprising VWF; and/or
(iii) expresses CD144, CD31, P-selectin, and ANG2 in similar quantities and in similar localization patterns as expressed by hPSC-EC comprising VWF.

14. The human endothelial cell of claim 12, wherein the hPSC-EC lacking VWF does not secrete VWF multimers.

15. The human endothelial cell of claim 10, wherein the hPSC-EC (i) expresses CD144, CD31, P-selectin, and ANG2; (ii) expresses VWF stored in WPBs; (iii) forms capillary-like structures; and (iv) secretes VWF multimers.

16. The human endothelial cell of claim 11, wherein the endothelial cell is capable of forming a cell line, a teratoma, a tissue, or an organoid.

17. A cell line or an organoid generated from the hPSC-EC of claim 16.

18. The cell line or organoid of claim 17, wherein the cell line or organoid models a feature of blood disorder selected from: thrombosis, deep vein thrombosis, blood clots, stroke, heart disease and von Willebrand disease.

19. A microfluidic culture device comprising the organoid of claim 17, wherein the microfluidic device is an organ-on-chip configured to represent VWF disease.

20. A high throughput method for identifying a therapeutic agent for treating a bleeding disorder, the method comprising:
contacting a plurality of candidate agents with cells obtained from the cell line or the organoid of claim 17, wherein an agent effective in:
(i) enhancing the expression levels of VWF;
(ii) restoring the expression of VWF;
(iii) restoring VWF-linked protein secretion, and/or
(iv) restoring VWF string formation,
is identified as the therapeutic agent effective for treating bleeding disorders.

21. The method of claim 20, wherein the therapeutic agent is selected from: a genetic agent, a component of the cell culture, or a small molecule.

22. The method of claim 21, wherein the genetic agent is selected from a vector comprising a polynucleotide encoding VWF protein, a subunit, or a derivative thereof, or a polynucleotide encoding a protein linked/associated with VWF secretion, a subunit, or a derivative thereof.

23. The method of claim 21, wherein the component of the cell culture comprises blood plasma.

24. The method of claim 21, wherein the small molecule is an agonist of VWF expression, and wherein the small molecule comprises DAPT.

25. The method of claim 20 further comprising assessing toxicity and/or pharmacokinetics of the therapeutic agent.

26. The method of claim 20, wherein the bleeding disorder is selected from: thrombosis, deep vein thrombosis, blood clots, stroke, heart disease and von Willebrand disease.

27. A method of generating one or more human pluripotent stem cell lines comprising the steps of:
a) identifying at least one protein linked to VWF protein secretion in a biological sample;
b) contacting a plurality of hPSCs with an agent effective in suppressing or deleting at least one gene sequence or a portion thereof encoding the identified protein or a subunit thereof;
c) disrupting a VWF-linked protein secretion pathway in the plurality of hPSCs;
d) identifying modified hPSCs with disrupted VWF-linked protein secretion pathway;
e) expanding the modified hPSCs to obtain a plurality of modified hPSCs as a clonal population; and
f) deriving a cell lineage or tissue from the modified hPSCs clonal population to obtain endothelial cells.

28. The method of claim 27 further comprising an additional step of stimulating endothelial cell specific organelles, wherein the specific organelles are Weibel-Palade bodies (WPBs).


SEQUENCE LISTING (14 sequences)

SEQ ID NO: 1 - DNA, 8442 bp, synthetic construct
  source 1..8442: mol_type = other DNA
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcacaggtcaatcacacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacgtccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgcctgcttctgcgacaccattgctgcctatgcccacgtgtgtgcccagcatggcaaggtggtgacctggaggacggccacattgtgcccccagagctgcgaggagaggaatctccgggagaacgggtatgagtgtgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggaggcctggtggtgcctcccacagatgccccggtgagccccaccactctgtatgtggaggacatctcggaaccgccgttgcacgatttctactgcagcaggctactggacctggtcttcctgctggatggctcctccaggctgtccgaggctgagtttgaagtgctgaaggcctttgtggtggacatgatggagcggctgcgcatctcccagaagtgggtccgcgtggccgtggtggagtaccacgacggctcccacgcctacatcgggctcaaggaccggaagcgaccgtcagagctgcggcgcattgccagccaggtgaagtatgcgggcagccaggtggcctccaccagcgaggtcttgaaatacacactgttccaaatcttcagcaagatcgaccgccctgaagcctcccgcatcgccctgctcctgatggccagccaggagccccaacggatgtcccggaactttgtccgctacgtccagggcctgaagaagaagaaggtcattgtgatcccggtgggcattgggccccatgccaacctcaagcagatccgcctcatcgagaagcaggcccctgagaacaaggccttcgtgctgagcagtgtggatgagctggagcagcaaagggacgagatcgttagctacctctgtgaccttgcccctgaagcccctcctcctactctgccccccgacatggcacaagtcactgtgggcccggggctcttgggggtttcgaccctggggcccaagaggaactccatggttctggatgtggcgttcgtcctggaaggatcggacaaaattggtgaagccgacttcaacaggagcaaggagttcatggaggaggtgattcagcggatggatgtgggccaggacagcatccacgtcacggtgctgcagtactcctacatggtgactgtggagtaccccttcagcgaggcacagtccaaaggggacatcctgcagcgggtgcgagagatccgctaccagggcggcaacaggaccaacactgggctggccctgcggtacctctctgaccacagcttcttggtcagccagggtgaccgggagcaggcgcccaacctggtctacatggtcaccggaaatcctgcctctgatgagatcaagaggctgcctggagacatccaggtggtgcccattggagtgggccctaatgccaacgtgcaggagctggagaggattggctggcccaatgcccctatcctcatccaggactttgagacgctcccccgagaggctcctgacctggtgctgcagaggtgctgctccggagaggggctgcagatccccaccctctcccctgcacctgactgcagccagcccctggacgtgatccttctcctggatggctcctccagtttcccagcttcttattttgatgaaatgaagagtttcgccaaggctttcatttcaaaagccaatatagggcctcgtctcactcaggtgtcagtgctgcagtatggaagcatcaccaccattgacgtgccatggaacgtggtcccggagaaagcccatttgctgagccttgtggacgtcatgcagcgggagggaggccccagccaaatcggggatgccttgggctttgctgtgcgatacttgacttcagaaatgcatggtgccaggccgggagcctcaaaggcggtggtcatcctggtcacggacgtctctgtggattcagtggatgcagcagctgatgccgccaggtccaacagagtgacagtgttccctattggaattggagatcgctacgatgcagcccagctacggatcttggcaggcccagcaggcgactccaacgtggtgaagctccagcgaatcgaagacctccctaccatggtcaccttgggcaattccttcctccacaaactgtgctctggatttgttaggatttgcatggatgaggatgggaatgagaagaggcccggggacgtctggaccttgccagaccagtgccacaccgtgacttgccagccagatggccagaccttgctgaagagtcatcgggtcaactgtgaccgggggctgaggccttcgtgccctaacagccagtcccctgttaaagtggaagagacctgtggctgccgctggacctgcccctgcgtgtgcacaggcagctccactcggcacatcgtgacctttgatgggcagaatttcaagctgactggcagctgttcttatgtcctatttcaaaacaaggagcaggacctggaggtgattctccataatggtgcctgcagccctggagcaaggcagggctgcatgaaatccatcgaggtgaagcacagtgccctctccgtcgagctgcacagtgacatggaggtgacggtgaatgggagactggtctctgttccttacgtgggtgggaacatggaagtcaacgtttatggtgccatcatgcatgaggtcagattcaatcaccttggtcacatcttcacattcactccacaaaacaatgagttccaactgcagctcagccccaagacttttgcttcaaagacgtatggtctgtgtgggatctgtgatgagaacggagccaatgacttcatgctgagggatggcacagtcaccacagactggaaaacacttgttcaggaatggactgtgcagcggccagggcagacgtgccagcccatcctggaggagcagtgtcttgtccccgacagctcccactgccaggtcctcctcttaccactgtttgctgaatgccacaaggtcctggctccagccacattctatgccatctgccagcaggacagttgccaccaggagcaagtgtgtgaggtgatcgcctcttatgcccacctctgtcggaccaacggggtctgcgttgactggaggacacctgatttctgtgctatgtcatgcccaccatctctggtctacaaccactgtgagcatggctgtccccggcactgtgatggcaacgtgagctcctgtggggaccatccctccgaaggctgtttctgccctccagataaagtcatgttggaaggcagctgtgtccctgaagaggcctgcactcagtgcattggtgaggatggagtccagcaccagttcctggaagcctgggtcccggaccaccagccctgtcagatctgcacatgcctcagcgggcggaaggtcaactgcacaacgcagccctgccccacggccaaagctcccacgtgtggcctgtgtgaagtagcccgcctccgccagaatgcagaccagtgctgccccgagtatgagtgtgtgtgtgacccagtgagctgtgacctgcccccagtgcctcactgtgaacgtggcctccagcccacactgaccaaccctggcgagtgcagacccaacttcacctgcgcctgcaggaaggaggagtgcaaaagagtgtccccaccctcctgccccccgcaccgtttgcccacccttcggaagacccagtgctgtgatgagtatgagtgtgcctgcaactgtgtcaactccacagtgagctgtccccttgggtacttggcctcaactgccaccaatgactgtggctgtaccacaaccacctgccttcccgacaaggtgtgtgtccaccgaagcaccatctaccctgtgggccagttctgggaggagggctgcgatgtgtgcacctgcaccgacatggaggatgccgtgatgggcctccgcgtggcccagtgctcccagaagccctgtgaggacagctgtcggtcgggcttcacttacgttctgcatgaaggcgagtgctgtggaaggtgcctgccatctgcctgtgaggtggtgactggctcaccgcggggggactcccagtcttcctggaagagtgtcggctcccagtgggcctccccggagaacccctgcctcatcaatgagtgtgtccgagtgaaggaggaggtctttatacaacaaaggaacgtctcctgcccccagctggaggtccctgtctgcccctcgggctttcagctgagctgtaagacctcagcgtgctgcccaagctgtcgctgtgagcgcatggaggcctgcatgctcaatggcactgtcattgggcccgggaagactgtgatgatcgatgtgtgcacgacctgccgctgcatggtgcagatgggggtcatctctggattcaagctggagtgcaggaagaccacctgcaacccctgccccctgggttacaaggaagaaaataacacaggtgaatgttgtgggagatgtttgcctacggcttgcaccattcagctaagaggaggacagatcatgacactgaagcgtgatgagacgctccaggatggctgtgatactcacttctgcaaggtcaatgagagaggagagtacttctgggagaagagggtcacaggctgcccaccctttgatgaacacaagtgtctggctgagggaggtaaaattatgaaaattccaggcacctgctgtgacacatgtgaggagcctgagtgcaacgacatcactgccaggctgcagtatgtcaaggtgggaagctgtaagtctgaagtagaggtggatatccactactgccagggcaaatgtgccagcaaagccatgtactccattgacatcaacgatgtgcaggaccagtgctcctgctgctctccgacacagacggagcccatgcaggtggccctgcactgcaccaatggctctgttgtgtaccatgaggttctcaatgccatggagtgcaaatgctcccccaggaagtgcagcaagtag

SEQ ID NO: 2 - RNA, 23 nt, synthetic construct
  source 1..23: mol_type = other RNA
aactcgcggcaggtcatccacgg

SEQ ID NO: 3 - RNA, 23 nt, synthetic construct
  source 1..23: mol_type = other RNA
gtcaatggtaccgtgacacaggg

SEQ ID NO: 4 - protein, 2813 aa, synthetic construct
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCQDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 5 - DNA, 51 bp, synthetic construct
  source 1..51: mol_type = other DNA
tttgtgcagaaggaactcgcggcaggtccgatgcagccttttcggaagtga

SEQ ID NO: 6 - DNA, 74 bp, synthetic construct
  source 1..74: mol_type = other DNA
tgcagaaggaactcgcggcaggtcattccacggcccgatgcagccttttcggaagtgacttcgtcaacaccttt

SEQ ID NO: 7 - DNA, 61 bp, synthetic construct
  source 1..61: mol_type = other DNA
gacatccatttgtttgtcaatggtaccgtgaccacagggggaccaaaggtaagccaacaat

SEQ ID NO: 8 - DNA, 61 bp, synthetic construct
  source 1..61: mol_type = other DNA
tttgtgcagaaggaactcgcggcaggtcatccacggcccgatgcagccttttcggaagtga

SEQ ID NO: 9 - DNA, 73 bp, synthetic construct
  source 1..73: mol_type = other DNA
tgcagaaggaactcgcggcaggtcatccacggcccgatgcagccttttcggaagtgacttcgtcaacaccttt

SEQ ID NO: 10 - DNA, 60 bp, synthetic construct
  source 1..60: mol_type = other DNA
gacatccatttgtttgtcaatggtaccgtgacacagggggaccaaaggtaagccaacaat

SEQ ID NO: 11 - DNA, 8432 bp, synthetic construct
  source 1..8432: mol_type = other DNA
atgattcctgccagatttgccggggtgctgcttgctctggccctcattttgccagggaccctttgtgcagaaggaactcgcggcaggtccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcacaggtcaatcacacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacgtccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgcctgcttctgcgacaccattgctgcctatgcccacgtgtgtgcccagcatggcaaggtggtgacctggaggacggccacattgtgcccccagagctgcgaggagaggaatctccgggagaacgggtatgagtgtgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggaggcctggtggtgcctcccacagatgccccggtgagccccaccactctgtatgtggaggacatctcggaaccgccgttgcacgatttctactgcagcaggctactggacctggtcttcctgctggatggctcctccaggctgtccgaggctgagtttgaagtgctgaaggcctttgtggtggacatgatggagcggctgcgcatctcccagaagtgggtccgcgtggccgtggtggagtaccacgacggctcccacgcctacatcgggctcaaggaccggaagcgaccgtcagagctgcggcgcattgccagccaggtgaagtatgcgggcagccaggtggcctccaccagcgaggtcttgaaatacacactgttccaaatcttcagcaagatcgaccgccctgaagcctcccgcatcgccctgctcctgatggccagccaggagccccaacggatgtcccggaactttgtccgctacgtccagggcctgaagaagaagaaggtcattgtgatcccggtgggcattgggccccatgccaacctcaagcagatccgcctcatcgagaagcaggcccctgagaacaaggccttcgtgctgagcagtgtggatgagctggagcagcaaagggacgagatcgttagctacctctgtgaccttgcccctgaagcccctcctcctactctgccccccgacatggcacaagtcactgtgggcccggggctcttgggggtttcgaccctggggcccaagaggaactccatggttctggatgtggcgttcgtcctggaaggatcggacaaaattggtgaagccgacttcaacaggagcaaggagttcatggaggaggtgattcagcggatggatgtgggccaggacagcatccacgtcacggtgctgcagtactcctacatggtgactgtggagtaccccttcagcgaggcacagtccaaaggggacatcctgcagcgggtgcgagagatccgctaccagggcggcaacaggaccaacactgggctggccctgcggtacctctctgaccacagcttcttggtcagccagggtgaccgggagcaggcgcccaacctggtctacatggtcaccggaaatcctgcctctgatgagatcaagaggctgcctggagacatccaggtggtgcccattggagtgggccctaatgccaacgtgcaggagctggagaggattggctggcccaatgcccctatcctcatccaggactttgagacgctcccccgagaggctcctgacctggtgctgcagaggtgctgctccggagaggggctgcagatccccaccctctcccctgcacctgactgcagccagcccctggacgtgatccttctcctggatggctcctccagtttcccagcttcttattttgatgaaatgaagagtttcgccaaggctttcatttcaaaagccaatatagggcctcgtctcactcaggtgtcagtgctgcagtatggaagcatcaccaccattgacgtgccatggaacgtggtcccggagaaagcccatttgctgagccttgtggacgtcatgcagcgggagggaggccccagccaaatcggggatgccttgggctttgctgtgcgatacttgacttcagaaatgcatggtgccaggccgggagcctcaaaggcggtggtcatcctggtcacggacgtctctgtggattcagtggatgcagcagctgatgccgccaggtccaacagagtgacagtgttccctattggaattggagatcgctacgatgcagcccagctacggatcttggcaggcccagcaggcgactccaacgtggtgaagctccagcgaatcgaagacctccctaccatggtcaccttgggcaattccttcctccacaaactgtgctctggatttgttaggatttgcatggatgaggatgggaatgagaagaggcccggggacgtctggaccttgccagaccagtgccacaccgtgacttgccagccagatggccagaccttgctgaagagtcatcgggtcaactgtgaccgggggctgaggccttcgtgccctaacagccagtcccctgttaaagtggaagagacctgtggctgccgctggacctgcccctgcgtgtgcacaggcagctccactcggcacatcgtgacctttgatgggcagaatttcaagctgactggcagctgttcttatgtcctatttcaaaacaaggagcaggacctggaggtgattctccataatggtgcctgcagccctggagcaaggcagggctgcatgaaatccatcgaggtgaagcacagtgccctctccgtcgagctgcacagtgacatggaggtgacggtgaatgggagactggtctctgttccttacgtgggtgggaacatggaagtcaacgtttatggtgccatcatgcatgaggtcagattcaatcaccttggtcacatcttcacattcactccacaaaacaatgagttccaactgcagctcagccccaagacttttgcttcaaagacgtatggtctgtgtgggatctgtgatgagaacggagccaatgacttcatgctgagggatggcacagtcaccacagactggaaaacacttgttcaggaatggactgtgcagcggccagggcagacgtgccagcccatcctggaggagcagtgtcttgtccccgacagctcccactgccaggtcctcctcttaccactgtttgctgaatgccacaaggtcctggctccagccacattctatgccatctgccagcaggacagttgccaccaggagcaagtgtgtgaggtgatcgcctcttatgcccacctctgtcggaccaacggggtctgcgttgactggaggacacctgatttctgtgctatgtcatgcccaccatctctggtctacaaccactgtgagcatggctgtccccggcactgtgatggcaacgtgagctcctgtggggaccatccctccgaaggctgtttctgccctccagataaagtcatgttggaaggcagctgtgtccctgaagaggcctgcactcagtgcattggtgaggatggagtccagcaccagttcctggaagcctgggtcccggaccaccagccctgtcagatctgcacatgcctcagcgggcggaaggtcaactgcacaacgcagccctgccccacggccaaagctcccacgtgtggcctgtgtgaagtagcccgcctccgccagaatgcagaccagtgctgccccgagtatgagtgtgtgtgtgacccagtgagctgtgacctgcccccagtgcctcactgtgaacgtggcctccagcccacactgaccaaccctggcgagtgcagacccaacttcacctgcgcctgcaggaaggaggagtgcaaaagagtgtccccaccctcctgccccccgcaccgtttgcccacccttcggaagacccagtgctgtgatgagtatgagtgtgcctgcaactgtgtcaactccacagtgagctgtccccttgggtacttggcctcaactgccaccaatgactgtggctgtaccacaaccacctgccttcccgacaaggtgtgtgtccaccgaagcaccatctaccctgtgggccagttctgggaggagggctgcgatgtgtgcacctgcaccgacatggaggatgccgtgatgggcctccgcgtggcccagtgctcccagaagccctgtgaggacagctgtcggtcgggcttcacttacgttctgcatgaaggcgagtgctgtggaaggtgcctgccatctgcctgtgaggtggtgactggctcaccgcggggggactcccagtcttcctggaagagtgtcggctcccagtgggcctccccggagaacccctgcctcatcaatgagtgtgtccgagtgaaggaggaggtctttatacaacaaaggaacgtctcctgcccccagctggaggtccctgtctgcccctcgggctttcagctgagctgtaagacctcagcgtgctgcccaagctgtcgctgtgagcgcatggaggcctgcatgctcaatggcactgtcattgggcccgggaagactgtgatgatcgatgtgtgcacgacctgccgctgcatggtgcagatgggggtcatctctggattcaagctggagtgcaggaagaccacctgcaacccctgccccctgggttacaaggaagaaaataacacaggtgaatgttgtgggagatgtttgcctacggcttgcaccattcagctaagaggaggacagatcatgacactgaagcgtgatgagacgctccaggatggctgtgatactcacttctgcaaggtcaatgagagaggagagtacttctgggagaagagggtcacaggctgcccaccctttgatgaacacaagtgtctggctgagggaggtaaaattatgaaaattccaggcacctgctgtgacacatgtgaggagcctgagtgcaacgacatcactgccaggctgcagtatgtcaaggtgggaagctgtaagtctgaagtagaggtggatatccactactgccagggcaaatgtgccagcaaagccatgtactccattgacatcaacgatgtgcaggaccagtgctcctgctgctctccgacacagacggagcccatgcaggtggccctgcactgcaccaatggctctgttgtgtaccatgaggttctcaatgccatggagtgcaaatgctcccccaggaagtgcagcaagtag

SEQ ID NO: 12 - DNA, 8443 bp, synthetic construct
  source 1..8443: mol_type = other DNA
atgattcctgccagatttgccggggtgctgcttgctctggccctcattttgccagggaccctttgtgcagaaggaactcgcggcaggtcattccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcacaggtcaatcacacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacgtccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgcctgcttctgcgacaccattgctgcctatgcccacgtgtgtgcccagcatggcaaggtggtgacctggaggacggccacattgtgcccccagagctgcgaggagaggaatctccgggagaacgggtatgagtgtgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggaggcctggtggtgcctcccacagatgccccggtgagccccaccactctgtatgtggaggacatctcggaaccgccgttgcacgatttctactgcagcaggctactggacctggtcttcctgctggatggctcctccaggctgtccgaggctgagtttgaagtgctgaaggcctttgtggtggacatgatggagcggctgcgcatctcccagaagtgggtccgcgtggccgtggtggagtaccacgacggctcccacgcctacatcgggctcaaggaccggaagcgaccgtcagagctgcggcgcattgccagccaggtgaagtatgcgggcagccaggtggcctccaccagcgaggtcttgaaatacacactgttccaaatcttcagcaagatcgaccgccctgaagcctcccgcatcgccctgctcctgatggccagccaggagccccaacggatgtcccggaactttgtccgctacgtccagggcctgaagaagaagaaggtcattgtgatcccggtgggcattgggccccatgccaacctcaagcagatccgcctcatcgagaagcaggcccctgagaacaaggccttcgtgctgagcagtgtggatgagctggagcagcaaagggacgagatcgttagctacctctgtgaccttgcccctgaagcccctcctcctactctgccccccgacatggcacaagtcactgtgggcccggggctcttgggggtttcgaccctggggcccaagaggaactccatggttctggatgtggcgttcgtcctggaaggatcggacaaaattggtgaagccgacttcaacaggagcaaggagttcatggaggaggtgattcagcggatggatgtgggccaggacagcatccacgtcacggtgctgcagtactcctacatggtgactgtggagtaccccttcagcgaggcacagtccaaaggggacatcctgcagcgggtgcgagagatccgctaccagggcggcaacaggaccaacactgggctggccctgcggtacctctctgaccacagcttcttggtcagccagggtgaccgggagcaggcgcccaacctggtctacatggtcaccggaaatcctgcctctgatgagatcaagaggctgcctggagacatccaggtggtgcccattggagtgggccctaatgccaacgtgcaggagctggagaggattggctggcccaatgcccctatcctcatccaggactttgagacgctcccccgagaggctcctgacctggtgctgcagaggtgctgctccggagaggggctgcagatccccaccctctcccctgcacctgactgcagccagcccctggacgtgatccttctcctggatggctcctccagtttcccagcttcttattttgatgaaatgaagagtttcgccaaggctttcatttcaaaagccaatatagggcctcgtctcactcaggtgtcagtgctgcagtatggaagcatcaccaccattgacgtgccatggaacgtggtcccggagaaagcccatttgctgagccttgtggacgtcatgcagcgggagggaggccccagccaaatcggggatgccttgggctttgctgtgcgatacttgacttcagaaatgcatggtgccaggccgggagcctcaaaggcggtggtcatcctggtcacggacgtctctgtggattcagtggatgcagcagctgatgccgccaggtccaacagagtgacagtgttccctattggaattggagatcgctacgatgcagcccagctacggatcttggcaggcccagcaggcgactccaacgtggtgaagctccagcgaatcgaagacctccctaccatggtcaccttgggcaattccttcctccacaaactgtgctctggatttgttaggatttgcatggatgaggatgggaatgagaagaggcccggggacgtctggaccttgccagaccagtgccacaccgtgacttgccagccagatggccagaccttgctgaagagtcatcgggtcaactgtgaccgggggctgaggccttcgtgccctaacagccagtcccctgttaaagtggaagagacctgtggctgccgctggacctgcccctgcgtgtgcacaggcagctccactcggcacatcgtgacctttgatgggcagaatttcaagctgactggcagctgttcttatgtcctatttcaaaacaaggagcaggacctggaggtgattctccataatggtgcctgcagccctggagcaaggcagggctgcatgaaatccatcgaggtgaagcacagtgccctctccgtcgagctgcacagtgacatggaggtgacggtgaatgggagactggtctctgttccttacgtgggtgggaacatggaagtcaacgtttatggtgccatcatgcatgaggtcagattcaatcaccttggtcacatcttcacattcactccacaaaacaatgagttccaactgcagctcagccccaagacttttgcttcaaagacgtatggtctgtgtgggatctgtgatgagaacggagccaatgacttcatgctgagggatggcacagtcaccacagactggaaaacacttgttcaggaatggactgtgcagcggccagggcagacgtgccagcccatcctggaggagcagtgtcttgtccccgacagctcccactgccaggtcctcctcttaccactgtttgctgaatgccacaaggtcctggctccagccacattctatgccatctgccagcaggacagttgccaccaggagcaagtgtgtgaggtgatcgcctcttatgcccacctctgtcggaccaacggggtctgcgttgactggaggacacctgatttctgtgctatgtcatgcccaccatctctggtctacaaccactgtgagcatggctgtccccggcactgtgatggcaacgtgagctcctgtggggaccatccctccgaaggctgtttctgccctccagataaagtcatgttggaaggcagctgtgtccctgaagaggcctgcactcagtgcattggtgaggatggagtccagcaccagttcctggaagcctgggtcccggaccaccagccctgtcagatctgcacatgcctcagcgggcggaaggtcaactgcacaacgcagccctgccccacggccaaagctcccacgtgtggcctgtgtgaagtagcccgcctccgccagaatgcagaccagtgctgccccgagtatgagtgtgtgtgtgacccagtgagctgtgacctgcccccagtgcctcactgtgaacgtggcctccagcccacactgaccaaccctggcgagtgcagacccaacttcacctgcgcctgcaggaaggaggagtgcaaaagagtgtccccaccctcctgccccccgcaccgtttgcccacccttcggaagacccagtgctgtgatgagtatgagtgtgcctgcaactgtgtcaactccacagtgagctgtccccttgggtacttggcctcaactgccaccaatgactgtggctgtaccacaaccacctgccttcccgacaaggtgtgtgtccaccgaagcaccatctaccctgtgggccagttctgggaggagggctgcgatgtgtgcacctgcaccgacatggaggatgccgtgatgggcctccgcgtggcccagtgctcccagaagccctgtgaggacagctgtcggtcgggcttcacttacgttctgcatgaaggcgagtgctgtggaaggtgcctgccatctgcctgtgaggtggtgactggctcaccgcggggggactcccagtcttcctggaagagtgtcggctcccagtgggcctccccggagaacccctgcctcatcaatgagtgtgtccgagtgaaggaggaggtctttatacaacaaaggaacgtctcctgcccccagctggaggtccctgtctgcccctcgggctttcagctgagctgtaagacctcagcgtgctgcccaagctgtcgctgtgagcgcatggaggcctgcatgctcaatggcactgtcattgggcccgggaagactgtgatgatcgatgtgtgcacgacctgccgctgcatggtgcagatgggggtcatctctggattcaagctggagtgcaggaagaccacctgcaacccctgccccctgggttacaaggaagaaaataacacaggtgaatgttgtgggagatgtttgcctacggcttgcaccattcagctaagaggaggacagatcatgacactgaagcgtgatgagacgctccaggatggctgtgatactcacttctgcaaggtcaatgagagaggagagtacttctgggagaagagggtcacaggctgcccaccctttgatgaacacaagtgtctggctgagggaggtaaaattatgaaaattccaggcacctgctgtgacacatgtgaggagcctgagtgcaacgacatcactgccaggctgcagtatgtcaaggtgggaagctgtaagtctgaagtagaggtggatatccactactgccagggcaaatgtgccagcaaagccatgtactccattgacatcaacgatgtgcaggaccagtgctcctgctgctctccgacacagacggagcccatgcaggtggccctgcactgcaccaatggctctgttgtgtaccatgaggttctcaatgccatggagtgcaaatgctcccccaggaagtgcagcaagtag

SEQ ID NO: 13 - DNA, 8443 bp, synthetic construct
  source 1..8443: mol_type = other DNA
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgaccacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcacaggtcaatcacacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacgtccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgcctgcttctgcgacaccattgctgcctatgcccacgtgtgtgcccagcatggcaaggtggtgacctggaggacggccacattgtgcccccagagctgcgaggagaggaatctccgggagaacgggtatgagtgtgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggaggcctggtggtgcctcccacagatgccccggtgagccccaccactctgtatgtggaggacatctcggaaccgccgttgcacgatttctactgcagcaggctactggacctggtcttcctgctggatggctcctccaggctgtccgaggctgagtttgaagtgctgaaggcctttgtggtggacatgatggagcggctgcgcatctcccagaagtgggtccgcgtggccgtggtggagtaccacgacggctcccacgcctacatcgggctcaaggaccggaagcgaccgtcagagctgcggcgcattgccagccaggtgaagtatgcgggcagccaggtggcctccaccagcgaggtcttgaaatacacactgttccaaatcttcagcaagatcgaccgccctgaagcctcccgcatcgccctgctcctgatggccagccaggagccccaacggatgtcccggaactttgtccgctacgtccagggcctgaagaagaagaaggtcattgtgatcccggtgggcattgggccccatgccaacctcaagcagatccgcctcatcgagaagcaggcccctgagaacaaggccttcgtgctgagcagtgtggatgagctggagcagcaaagggacgagatcgttagctacctctgtgaccttgcccctgaagcccctcctcctactctgccccccgacatggcacaagtcactgtgggcccggggctcttgggggtttcgaccctggggcccaagaggaactccatggttctggatgtggcgttcgtcctggaaggatcggacaaaattggtgaagccgacttcaacaggagcaaggagttcatggaggaggtgattcagcggatggatgtgggccaggacagcatccacgtcacggtgctgcagtactcctacatggtgactgtggagtaccccttcagcgaggcacagtccaaaggggacatcctgcagcgggtgcgagagatccgctaccagggcggcaacaggaccaacactgggctggccctgcggtacctctctgaccacagcttcttggtcagccagggtgaccgggagcaggcgcccaacctggtctacatggtcaccggaaatcctgcctctgatgagatcaagaggctgcctggagacatccaggtggtgcccattggagtgggccctaatgccaacgtgcaggagctggagaggattggctggcccaatgcccctatcctcatccaggactttgagacgctcccccgagaggctcctgacctggtgctgcagaggtgctgctccggagaggggctgcagatccccaccctctcccctgcacctgactgcagccagcccctggacgtgatccttctcctggatggctcctccagtttcccagcttcttattttgatgaaatgaagagtttcgccaaggctttcatttcaaaagccaatatagggcctcgtctcactcaggtgtcagtgctgcagtatggaagcatcaccaccattgacgtgccatggaacgtggtcccggagaaagcccatttgctgagccttgtggacgtcatgcagcgggagggaggccccagccaaatcggggatgccttgggctttgctgtgcgatacttgacttcagaaatgcatggtgccaggccgggagcctcaaaggcggtggtcatcctggtcacggacgtctctgtggattcagtggatgcagcagctgatgccgccaggtccaacagagtgacagtgttccctattggaattggagatcgctacgatgcagcccagctacggatcttggcaggcccagcaggcgactccaacgtggtgaagctccagcgaatcgaagacctccctaccatggtcaccttgggcaattccttcctccacaaactgtgctctggatttgttaggatttgcatggatgaggatgggaatgagaagaggcccggggacgtctggaccttgccagaccagtgccacaccgtgacttgccagccagatggccagaccttgctgaagagtcatcgggtcaactgtgaccgggggctgaggccttcgtgccctaacagccagtcccctgttaaagtggaagagacctgtggctgccgctggacctgcccctgcgtgtgcacaggcagctccactcggcacatcgtgacctttgatgggcagaatttcaagctgactggcagctgttcttatgtcctatttcaaaacaaggagcaggacctggaggtgattctccataatggtgcctgcagccctggagcaaggcagggctgcatgaaatccatcgaggtgaagcacagtgccctctccgtcgagctgcacagtgacatggaggtgacggtgaatgggagactggtctctgttccttacgtgggtgggaacatggaagtcaacgtttatggtgccatcatgcatgaggtcagattcaatcaccttggtcacatcttcacattcactccacaaaacaatgagttccaactgcagctcagccccaagacttttgcttcaaagacgtatggtctgtgtgggatctgtgatgagaacggagccaatgacttcatgctgagggatggcacagtcaccacagactggaaaacacttgttcaggaatggactgtgcagcggccagggcagacgtgccagcccatcctggaggagcagtgtcttgtccccgacagctcccactgccaggtcctcctcttaccactgtttgctgaatgccacaaggtcctggctccagccacattctatgccatctgccagcaggacagttgccaccaggagcaagtgtgtgaggtgatcgcctcttatgcccacctctgtcggaccaacggggtctgcgttgactggaggacacctgatttctgtgctatgtcatgcccaccatctctggtctacaaccactgtgagcatggctgtccccggcactgtgatggcaacgtgagctcctgtggggaccatccctccgaaggctgtttctgccctccagataaagtcatgttggaaggcagctgtgtccctgaagaggcctgcactcagtgcattggtgaggatggagtccagcaccagttcctggaagcctgggtcccggaccaccagccctgtcagatctgcacatgcctcagcgggcggaaggtcaactgcacaacgcagccctgccccacggccaaagctcccacgtgtggcctgtgtgaagtagcccgcctccgccagaatgcagaccagtgctgccccgagtatgagtgtgtgtgtgacccagtgagctgtgacctgcccccagtgcctcactgtgaacgtggcctccagcccacactgaccaaccctggcgagtgcagacccaacttcacctgcgcctgcaggaaggaggagtgcaaaagagtgtccccaccctcctgccccccgcaccgtttgcccacccttcggaagacccagtgctgtgatgagtatgagtgtgcctgcaactgtgtcaactccacagtgagctgtccccttgggtacttggcctcaactgccaccaatgactgtggctgtaccacaaccacctgccttcccgacaaggtgtgtgtccaccgaagcaccatctaccctgtgggccagttctgggaggagggctgcgatgtgtgcacctgcaccgacatggaggatgccgtgatgggcctccgcgtggcccagtgctcccagaagccctgtgaggacagctgtcggtcgggcttcacttacgttctgcatgaaggcgagtgctgtggaaggtgcctgccatctgcctgtgaggtggtgactggctcaccgcggggggactcccagtcttcctggaagagtgtcggctcccagtgggcctccccggagaacccctgcctcatcaatgagtgtgtccgagtgaaggaggaggtctttatacaacaaaggaacgtctcctgcccccagctggaggtccctgtctgcccctcgggctttcagctgagctgtaagacctcagcgtgctgcccaagctgtcgctgtgagcgcatggaggcctgcatgctcaatggcactgtcattgggcccgggaagactgtgatgatcgatgtgtgcacgacctgccgctgcatggtgcagatgggggtcatctctggattcaagctggagtgcaggaagaccacctgcaacccctgccccctgggttacaaggaagaaaataacacaggtgaatgttgtgggagatgtttgcctacggcttgcaccattcagctaagaggaggacagatcatgacactgaagcgtgatgagacgctccaggatggctgtgatactcacttctgcaaggtcaatgagagaggagagtacttctgggagaagagggtcacaggctgcccaccctttgatgaacacaagtgtctggctgagggaggtaaaattatgaaaattccaggcacctgctgtgacacatgtgaggagcctgagtgcaacgacatcactgccaggctgcagtatgtcaaggtgggaagctgtaagtctgaagtagaggtggatatccactactgccagggcaaatgtgccagcaaagccatgtactccattgacatcaacgatgtgcaggaccagtgctcctgctgctctccgacacagacggagcccatgcaggtggccctgcactgcaccaatggctctgttgtgtaccatgaggttctcaatgccatggagtgcaaatgctcccccaggaagtgcagcaagtag

SEQ ID NO: 14 - DNA, 13843 bp, synthetic construct
  source 1..13843: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgagctcggatccgctagcaggcgcgccatgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcacaggtcaatcacacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacgtccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtgccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgcctgcttctgcgacaccattgctgcctatgcccacgtgtgtgcccagcatggcaaggtggtgacctggaggacggccacattgtgcccccagagctgcgaggagaggaatctccgggagaacgggtatgagtgtgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggaggcctggtggtgcctcccacagatgccccggtgagccccaccactctgtatgtggaggacatctcggaaccgccgttgcacgatttctactgcagcaggctactggacctggtcttcctgctggatggctcctccaggctgtccgaggctgagtttgaagtgctgaaggcctttgtggtggacatgatggagcggctgcgcatctcccagaagtgggtccgcgtggccgtggtggagtaccacgacggctcccacgcctacatcgggctcaaggaccggaagcgaccgtcagagctgcggcgcattgccagccaggtgaagtatgcgggcagccaggtggcctccaccagcgaggtcttgaaatacacactgttccaaatcttcagcaagatcgaccgccctgaagcctcccgcatcgccctgctcctgatggccagccaggagccccaacggatgtcccggaactttgtccgctacgtccagggcctgaagaagaagaaggtcattgtgatcccggtgggcattgggccccatgccaacctcaagcagatccgcctcatcgagaagcaggcccctgagaacaaggccttcgtgctgagcagtgtggatgagctggagcagcaaagggacgagatcgttagctacctctgtgaccttgcccctgaagcccctcctcctactctgccccccgacatggcacaagtcactgtgggcccggggctcttgggggtttcgaccctggggcccaagaggaactccatggttctggatgtggcgttcgtcctggaaggatcggacaaaattggtgaagccgacttcaacaggagcaaggagttcatggaggaggtgattcagcggatggatgtgggccaggacagcatccacgtcacggtgctgcagtactcctacatggtgaccgtggagtaccccttcagcgaggcacagtccaaaggggacatcctgcagcgggtgcgagagatccgctaccagggcggcaacaggaccaacactgggctggccctgcggtacctctctgaccacagcttcttggtcagccagggtgaccgggagcaggcgcccaacctggtctacatggtcaccggaaatcctgcctctgatgagatcaagaggctgcctggagacatccaggtggtgcccattggagtgggccctaatgccaacgtgcaggagctggagaggattggctggcccaatgcccctatcctcatccaggactttgagacgctcccccgagaggctcctgacctggtgctgcagaggtgctgctccggagaggggctgcagatccccaccctctcccctgcacctgactgcagccagcccctggacgtgatccttctcctggatggctcctccagtttcccagcttcttattttgatgaaatgaagagtttcgccaaggctttcatttcaaaagccaatatagggcctcgtctcactcaggtgtcagtgctgcagtatggaagcatcaccaccattgacgtgccatggaacgtggtcccggagaaagcccatttgctgagccttgtggacgtcatgcagcgggagggaggccccagccaaatcggggatgccttgggctttgctgtgcgatacttgacttcagaaatgcatggtgccaggccgggagcctcaaaggcggtggtcatcctggtcacggacgtctctgtggattcagtggatgcagcagctgatgccgccaggtccaacagagtgacagtgttccctattggaattggagatcgctacgatgcagcccagctacggatcttggcaggcccagcaggcgactccaacgtggtgaagctccagcgaatcgaagacctccctaccatggtcaccttgggcaattccttcctccacaaactgtgctctggatttgttaggatttgcatggatgaggatgggaatgagaagaggcccggggacgtctggaccttgccagaccagtgccacaccgtgacttgccagccagatggccagaccttgctgaagagtcatcgggtcaactgtgaccgggggctgaggccttcgtgccctaacagccagtcccctgttaaagtggaagagacctgtggctgccgctggacctgcccctgcgtgtgcacaggcagctccactcggcacatcgtgacctttgatgggcagaatttcaagctgactggcagctgttcttatgtcctatttcaaaacaaggagcaggacctggaggtgattctccataatggtgcctgcagccctggagcaaggcagggctgcatgaaatccatcgaggtgaagcacagtgccctctccgtcgagctgcacagtgacatggaggtgacggtgaatgggagactggtctctgttccttacgtgggtgggaacatggaagtcaacgtttatggtgccatcatgcatgaggtcagattcaatcaccttggtcacatcttcacattcactccacaaaacaatgagttccaactgcagctcagccccaagacttttgcttcaaagacgtatggtctgtgtgggatctgtgatgagaacggagccaatgacttcatgctgagggatggcacagtcaccacagactggaaaacacttgttcaggaatggactgtgcagcggccagggcagacgtgccagcccatcctggaggagcagtgtcttgtccccgacagctcccactgccaggtcctcctcttaccactgtttgctgaatgccacaaggtcctggctccagccacattctatgccatctgccagcaggacagttgccaccaggagcaagtgtgtgaggtgatcgcctcttatgcccacctctgtcggaccaacggggtctgcgttgactggaggacacctgatttctgtgctatgtcatgcccaccatctctggtctacaaccactgtgagcatggctgtccccggcactgtgatggcaacgtgagctcctgtggggaccatccctccgaaggctgtttctgccctccagataaagtcatgttggaaggcagctgtgtccctgaagaggcctgcactcagtgcattggtgaggatggagtccagcaccagttcctggaagcctgggtcccggaccaccagccctgtcagatctgcacatgcctcagcgggcggaaggtcaactgcacaacgcagccctgccccacggccaaagctcccacgtgtggcctgtgtgaagtagcccgcctccgccagaatgcagaccagtgctgccccgagtatgagtgtgtgtgtgacccagtgagctgtgacctgcccccagtgcctcactgtgaacgtggcctccagcccacactgaccaaccctggcgagtgcagacccaacttcacctgcgcctgcaggaaggaggagtgcaaaagagtgtccccaccctcctgccccccgcaccgtttgcccacccttcggaagacccagtgctgtgatgagtatgagtgtgcctgcaactgtgtcaactccacagtgagctgtccccttgggtacttggcctcaactgccaccaatgactgtggctgtaccacaaccacctgccttcccgacaaggtgtgtgtccaccgaagcaccatctaccctgtgggccagttctgggaggagggctgcgatgtgtgcacctgcaccgacatggaggatgccgtgatgggcctccgcgtggcccagtgctcccagaagccctgtgaggacagctgtcggtcgggcttcacttacgttctgcatgaaggcgagtgctgtggaaggtgcctgccatctgcctgtgaggtggtgactggctcaccgcggggggactcccagtcttcctggaagagtgtcggctcccagtgggcctccccggagaacccctgcctcatcaatgagtgtgtccgagtgaaggaggaggtctttatacaacaaaggaacgtctcctgcccccagctggaggtccctgtctgcccctcgggctttcagctgagctgtaagacctcagcgtgctgcccaagctgtcgctgtgagcgcatggaggcctgcatgctcaatggcactgtcattgggcccgggaagactgtgatgatcgatgtgtgcacgacctgccgctgcatggtgcaggtgggggtcatctctggattcaagctggagtgcaggaagaccacctgcaacccctgccccctgggttacaaggaagaaaataacacaggtgaatgttgtgggagatgtttgcctacggcttgcaccattcagctaagaggaggacagatcatgacactgaagcgtgatgagacgctccaggatggctgtgatactcacttctgcaaggtcaatgagagaggagagtacttctgggagaagagggtcacaggctgcccaccctttgatgaacacaagtgtctggctgagggaggtaaaattatgaaaattccaggcacctgctgtgacacatgtgaggagcctgagtgcaacgacatcactgccaggctgcagtatgtcaaggtgggaagctgtaagtctgaagtagaggtggatatccactactgccagggcaaatgtgccagcaaagccatgtactccattgacatcaacgatgtgcaggaccagtgctcctgctgctctccgacacggacggagcccatgcaggtggccctgcactgcaccaatggctctgttgtgtaccatgaggttctcaatgccatggagtgcaaatgctcccccaggaagtgcagcaagggcagcggccatcaccatcaccatcacgcgtaagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc